SEQUENCE LISTING (49 sequences)

SEQ ID NO: 1 - protein, 253 aa, Artificial Sequence
  Amb a 1 variant
ANPKEGTLRFAAAQNRPLWIIFKNDMVINLNQELVVNSDKTIDGRGVKVEIINGGLTLMNVKNIIIHNINIHDVKVLPGGMIKSNDGPPILRQASDGDTINVAGSSQIWIDHCSLSKSFDGLVDVTLGSTHVTISNCKFTQQSKAILLGADDTHVQDKGMLATVAFNMFTDNVDQRMPRCRFGFFQVVNNNYDRWGTYAIGGSSAPTILCQGNRFLAPDDQIKKNVLARTGTGAAESMAWNWRSDKDLLENGA

SEQ ID NO: 2 - protein, 254 aa, Artificial Sequence
  Cry j 1 variant
APGTLRYGATRDRPLWIIFSGNMNIKLKMPMYIAGYKTFDGRGAQVYIGNGGPCVFIKRVSNVIIHGLHLYGCSTSVLGNVLINESFGVEPVHPQDGDALTLRTATNIWIDHNSFSNSSDGLVDVTLSSTGVTISNNLFFNHHKVMLLGHDDAYSDDKSMKVTVAFNQFGPNCGQRMPRARYGLVHVANNNYDPWTIYAIGGSSNPTILSEGNSFTAPNESYKKQVTIRIGCKTSSSCSNWVWQSTQDVFYNGA

SEQ ID NO: 3 - protein, 254 aa, Artificial Sequence
  Cup a 1 variant
TPGTLRYGATREKALWIIFSQNMNIKLQMPLYVAGYKTIDGRGAVVHLGNGGPCLFMRKASHVILHGLHIHGCNTSVLGDVLVSESIGVEPVHAQDGDAITMRNVTNAWIDHNSLSDCSDGLIDVTLGSTGITISNNHFFNHHKVMLLGHDDTYDDDKSMKVTVAFNQFGPNAGQRMPRARYGLVHVANNNYDQWNIYAIGGSSNPTILSEGNSFTAPNESYKKEVTKRIGCETTSACANWVWRSTRDAFTNGA

SEQ ID NO: 4 - protein, 254 aa, Artificial Sequence
  Jun a 1 variant
TPGTLRYGATREKALWIIFSQNMNIKLKMPLYVAGHKTIDGRGADVHLGNGGPCLFMRKVSHVILHSLHIHGCNTSVLGDVLVSESIGVEPVHAQDGDAITMRNVTNAWIDHNSLSDCSDGLIDVTLGSTGITISNNHFFNHHKVMLLGHDDTYDDDKSMKVTVAFNQFGPNAGQRMPRARYGLVHVANNNYDPWNIYAIGGSSNPTILSEGNSFTAPSESYKKEVTKRIGCESPSACANWVWRSTRDAFINGA

SEQ ID NO: 5 - protein, 254 aa, Artificial Sequence
  Cha o 1 variant
APGTLRYGATRERSLWIIFSKNLNIKLNMPLYIAGNKTIDGRGAEVHIGNGGPCLFMRTVSHVILHGLNIHGCNTSVSGNVLISEASGVVPVHAQDGDAITMRNVTDVWIDHNSLSDSSDGLVDVTLASTGVTISNNHFFNHHKVMLLGHSDIYSDDKSMKVTVAFNQFGPNAGQRMPRARYGLIHVANNNYDPWSIYAIGGSSNPTILSEGNSFTAPNDSDKKEVTRRVGCESPSTCANWVWRSTQDSFNNGA

SEQ ID NO: 6 - protein, 263 aa, Artificial Sequence
  Art v 6 variant
ANPKPGTLRCGVTQDKPLWIIFKKDMVIKLKHELVINKDKTIDGRGANVEITCGGLTIHNVCNVIIHNIHIHDIKVTEGGIIKATDAKPGHRHKSDGDGICVAGSSKIWIDHCTLSHGPDGLIDVTLGSTAVTISNCKFSHHQKILLLGADNSHVDDKKMHVTVAFNRFAEACDQRMPRCRFGFFQVVNNDYTSWGTYAIGGSANPTILSQGNRFHAPNDPMKKNVLVRADAPHTESMKWNWRSEKDLLENGAIFVASGCDPH

SEQ ID NO: 7 - DNA, 759 bp, Artificial Sequence
  Amb a 1 variant
gcaaatccaaaagaaggcacactccggtttgctgccgcccaaaacaggcccttgtggatcatttttaaaaatgatatggtgattaatttgaatcaagagcttgtcgtaaacagcgacaagaccatcgatggccgaggagtgaaagttgaaatcattaacggaggtctcaccctcatgaatgtcaagaatataatcattcataacataaatatccatgatgttaaagtgcttccaggaggcatgattaagtccaacgatggtccaccaattttaagacaagcaagtgatggtgatactataaatgttgctggtagttcccaaatatggatagaccattgctcactcagcaagtctttcgatgggctggtcgatgtcaccctcggtagcacacacgtgaccatttccaactgcaaattcacccaacagtcaaaagcaatattgttgggggcagatgacacccatgttcaagataaaggaatgctagccacggtcgctttcaacatgttcaccgataacgttgaccaaagaatgcctagatgtcgatttgggtttttccaagttgttaacaacaactacgacagatggggaacgtacgccataggtggtagctcggccccaactatactctgccaagggaacagattcttggcccctgatgatcagatcaagaaaaatgtcctagcgaggactggtacaggcgctgctgagtcgatggcgtggaactggagatctgataaagacttgcttgaaaatggtgct

SEQ ID NO: 8 - DNA, 759 bp, Artificial Sequence
  Amb a 1 variant
gcaaacccgaaagaaggtacgttacggtttgccgccgcccagaaccgtcccttatggattatttttaaaaacgatatggttattaacttaaaccaggagcttgttgtaaacagcgacaaaaccattgatggtcgaggtgttaaagtggaaattattaacggtggcttaaccttaatgaacgttaaaaacatcattattcataacatcaacattcatgatgtgaaagttcttccgggtggtatgattaaatcgaacgatggcccgccgattttacgtcaggcaagcgatggcgatacgatcaacgtggccggcagctcgcagatctggatcgaccattgctcgttaagcaaatcgtttgatggtctagtggatgtgaccttaggcagcacgcacgttaccatttcgaactgcaaatttacccagcaatcgaaagcaatcttattaggtgcagatgacacccatgtgcaggataaaggtatgctcgccacggttgcctttaacatgtttaccgataacgtggaccagcgtatgcctcgttgccgatttggtttttttcaggtggtgaacaacaactacgaccgttggggtacttacgccatcggcggcagctcggccccgacgatcttatgccagggtaaccgttttttagcccctgatgatcaaattaaaaaaaacgttctcgctcgtacgggcacgggtgccgccgagtcgatggcttggaactggcgttcggataaagacttacttgaaaacggcgcc

SEQ ID NO: 9 - DNA, 762 bp, Artificial Sequence
  Cry j 1 variant
gcaccaggaactctgcgctatggagcaacccgagataggcccctgtggataattttcagtgggaatatgaatataaagctcaaaatgcctatgtacattgctgggtataagacttttgatggcaggggagcacaagtttatattggcaatggcggtccctgtgtgtttatcaagagagttagcaatgttatcatacacggtttgtatctgtacggctgtagtactagtgttttggggaatgttttgataaacgagagttttggggtggagcctgttcatcctcaggatggcgatgctcttactctgcgcactgctacaaatatttggattgatcataattctttctccaattcttctgatggtctggtcgatgtcactcttacttcgactggagttactatttcaaacaatctttttttcaaccatcataaagtgatgttgttagggcatgatgatgcatatagtgatgacaaatccatgaaggtgacagtggcgttcaatcaatttggacctaactgtggacaaagaatgcccagggcacgatatggacttgtacatgttgcaaacaataattatgacccatggactatatatgcaattggtgggagttcaaatccaaccattctaagtgaagggaatagtttcactgcaccaaatgagagctacaagaagcaagtaaccatacgtattggatgcaaaacatcatcatcttgttcaaattgggtgtggcaatctacacaagatgttttttataatggagct

SEQ ID NO: 10 - DNA, 762 bp, Artificial Sequence
  Cry j 1 variant
ccgggcaccctgcggtatggcgccacccgagataggcctctgtggatcatttttagcggtaacatgaacatcaaattaaaaatgccgatgtacattgccggttataaaacctttgatggtaggggcgcccaggtgtatattggtaacggtggtccttgcgtgtttatcaaacgtgtgagtaacgtgatcatccacggtctgtatctgtacggttgcagcaccagcgtgctgggtaacgtgctgatcaatgaaagctttggtgtggaaccggtgcatccgcaggatggtgatgccctgaccctgcggaccgccaccaacatttggattgatcataactcgttttcgaactcgtcggatggtctggttgatgttaccctgacctcgaccggcgtgaccatttcgaataacctgttttttaatcatcataaagtgatgctgttgggtcatgatgatgcctatagcgatgacaaatcgatgaaagtgaccgtggcttttaaccagtttggcccgaattgcggccagcgtatgcctagggcccgatatggcctggttcatgtggccaataacaactatgacccgtggaccatctatgccattggtggtagctcgaacccgaccattctcagcgaaggtaacagctttaccgccccgaacgaaagttacaaaaaacaggttaccatccgaattggctgcaaaacctcgtcgtcgtgctcgaactgggtgtggcagtcgacccaggatgtgttttataacggcgcctat

SEQ ID NO: 11 - DNA, 762 bp, Artificial Sequence
  Cup a 1 variant
acaccaggaactttgcgctatggagcaacaagagaaaaagcactttggataattttctctcagaatatgaatataaagctccagatgcctttgtatgttgctggatataagactattgacggcaggggagcagttgttcatcttggcaatggcggtccctgtctgtttatgaggaaagcgagccatgttattctccatggtttgcatatacacggttgtaatacgagtgttttgggggatgttttggtaagtgagtccattggtgtggagcctgttcatgctcaggatggggacgccattactatgcggaatgttacaaatgcttggattgatcataattctctctccgattgttctgatggtcttatcgatgttacacttggttccactggaattactatctccaacaatcacttcttcaaccatcataaagtgatgttgttaggacatgatgatacatatgatgatgacaaatctatgaaagtgacagtggccttcaatcaatttggacccaatgctgggcaaagaatgccaagggcacgatatggacttgtacatgttgcaaacaataattatgatcaatggaatatatatgctattggtgggagttcaaatccaaccattctaagtgaagggaatagtttcactgctccaaatgagagctacaaaaaggaagtaacaaagcgtatagggtgtgaaacaacatcagcttgtgcgaactgggtgtggagatccacacgagatgcttttactaatggagct

SEQ ID NO: 12 - DNA, 762 bp, Artificial Sequence
  Cup a 1 variant
acccctggcaccttaaggtatggcgcaacccgagaaaaagcattatggatcattttttcgcaaaacatgaacatcaaactccaaatgcctttatatgtggccggctataaaaccattgacggacgaggcgcagtggtgcatttaggaaacggaggtccctgcctattcatgcgaaaagctagccatgtgattctccacggtttacatatccacggttgcaacacaagcgtgttaggtgatgtgttagtaagcgaatcgattggtgtggaacctgtgcatgcccaagatggtgacgctattaccatgcgaaacgtgaccaacgcctggattgatcataactcgctctcggattgctcggatggtttaatcgatgtgaccttaggttcgaccggcattaccatctcgaataaccacttttttaatcatcataaagtgatgttactaggccatgatgatacctatgatgatgacaaatcgatgaaagtgaccgtggcttttaaccaattcggccccaacgccggtcaacgaatgcctcgggcacgatatggcttagtacatgtggcaaataacaactatgatcaatggaacatctatgccattggtggtagctcgaaccctacgattctaagcgaaggtaacagctttaccgcccctaacgaaagctacaaaaaagaagtaaccaaaaggatcggttgcgaaaccacctcggcctgcgctaattgggtgtggcgatcgacccgagatgccttcaccaacggcgcc

SEQ ID NO: 13 - DNA, 759 bp, Artificial Sequence
  Art v 6 variant
gcaaatccaaagccagggacacttcgttgtggtgtcacccaagataaacctttgtggatcatctttaagaaagatatggtcataaaacttaaacacgagcttgtgataaacaaagacaagacaattgatggaagaggtgcaaatgttgagatcacttgtggcggtctcaccattcacaacgtttgcaatgtgatcattcataacattcacatacatgatattaaagtaaccgaaggtggaattattaaggcaacggacgctaaaccaggacatagacataagagcgacggagatggtatttgtgttgctggttcttcaaagatatggatcgatcattgcacacttagtcatggtccagatggccttattgatgtcacgttgggtagcacagccgttaccatttccaattgcaaatttagccatcaccaaaaaattctattactcggagcagacaattcacatgtagacgataaaaaaatgcatgtcacagtagcattcaacaggttcgcagaagcatgtgatcaaagaatgccacgatgtcgatttggatttttccaagttgttaacaatgactacaccagctggggaacgtacgccattggtggtagtgccaatcctactatccttagccaaggcaaccgattccatgctccgaatgacccaatgaagaaaaatgtgttggtgagggctgatgcaccacatacagagtcaatgaagtggaattggagatctgagaaagacttgttagaaaatggagct

SEQ ID NO: 14 - DNA, 759 bp, Artificial Sequence
  Art v 6 variant
gccaatcctaaacctggaacgttacggtgcggcgtcacccaagataaaccgttatggatcatctttaaaaaagatatggtcatcaaattaaaacacgagttagtaatcaataaagacaaaacgattgatggccgaggcgccaatgtggagatcacgtgcggtggccttaccattcacaatgtgtgcaatgtaatcattcataatattcacatccatgatattaaagtaaccgaaggcggcattattaaagccactgacgcgaaacctggccatcgacataaaagtgacggcgatggcatttgcgtggcgggctcgtcgaaaatctggatcgatcattgcacgttaagtcatggccctgatggtttaattgatgtcactttaggcagtacggcagtgaccatttccaattgcaaatttagtcatcaccaaaaaattctactacttggcgccgacaattcgcatgtagacgataaaaaaatgcatgtcacggtagccttcaatcgattcgccgaagcctgcgatcaacgaatgcctcgatgccgatttggctttttccaagtggtgaataatgactacaccagttggggcacttacgcaattggcggcagtgcaaatccgacgatcttaagtcaaggtaatcgattccatgcgcccaatgaccctatgaaaaaaaatgtattagtacgagcggatgcccctcatacggagtcgatgaaatggaattggcgatcggagaaagacttactcgaaaatggcgcg

SEQ ID NO: 15 - DNA, 762 bp, Artificial Sequence
  Jun a 1 variant
acaccaggaactttgcgctatggagcaacaagagaaaaagcactttggatcattttctctcagaatatgaatataaagctcaagatgcctttgtatgttgctggacataagactattgacggcaggggagcagatgttcatcttggcaacggcggtccctgtctgtttatgaggaaagtgagccatgttattctccatagtttgcatatacacggttgtaatacgagtgttttgggggatgttttggtaagtgagtctattggggtggagcctgttcatgctcaggatggggacgccattactatgcgcaatgttacaaatgcttggattgatcataattctctctccgattgttctgatggtcttatcgatgttacgcttggctccactggaattactatctccaacaatcacttcttcaaccatcataaagtgatgttgttaggacatgatgatacatatgacgatgacaaatctatgaaagtgacagtggcgttcaatcaatttggacctaatgctgggcaaagaatgccaagggcacgatatggacttgtacatgtcgcaaacaataattatgatccatggaatatatatgctattggtgggagttcaaatccaaccattctaagtgaagggaatagtttcactgccccaagtgagagctacaagaaggaagtaacaaagcgtatagggtgtgaatcaccatcagcttgtgcgaactgggtgtggagatctacacgagatgcttttattaatggagct

SEQ ID NO: 16 - DNA, 762 bp, Artificial Sequence
  Jun a 1 variant
accccgggcaccttaaggtacggcgccacccgagagaaagccttatggatcatttttagccaaaacatgaacatcaaactcaaaatgccgttatacgtggccggccataaaaccattgacggacgaggcgccgatgtgcatttaggaaatggaggaccctgcctatttatgcgaaaagttagccatgtgattctccatagcttacatatccacggatgcaacacgagcgtgttaggtgatgtgttagttagcgagagcattggtgttgagccggtgcatgcccaagatggtgacgctattaccatgaggaacgtgaccaacgcctggattgatcataacagcctctcggattgcagcgatggattaatcgatgtgacgttaggatcgaccggcattaccatctcgaataaccacttttttaatcatcataaagttatgttactcggccatgatgatacctacgacgatgacaaaagcatgaaagttaccgttgcttttaaccaatttggcccgaacgccggtcaacgaatgccgcgagcccgatacggcttagttcatgtagccaataacaactacgatccgtggaacatctacgccattggaggtagctcgaacccgacgattctaagcgagggtaacagctttaccgctccgagcgagagctacaaaaaagaggttaccaaacggatcggttgcgagtcgccgtcggcctgcgctaattgggtttggcgatcgacccgagatgcctttattaacggcgcc

SEQ ID NO: 17 - DNA, 762 bp, Artificial Sequence
  Cha o 1 variant
gcaccaggtactctgcgctacggggcaacacgagaaaggtcactgtggatcattttctctaagaatctgaacataaagctcaacatgcctttgtacattgctgggaataagaccattgatggcagaggagcagaagttcatattggcaatggtggtccctgtctgtttatgaggacagtgagccatgttattctacacggattgaatatacacggctgtaatacaagtgtttcggggaatgttttgataagcgaggcttctggagtggtgcctgttcatgctcaggatggcgacgccattactatgcgcaatgttacagatgtttggattgatcataattctctctccgattcttctgatggtcttgtcgatgttacacttgcttccaccggagttactatttccaacaatcattttttcaaccatcataaagtgatgttgttagggcatagtgatatatatagtgatgacaaaagtatgaaggtgacagtggcattcaatcaatttggacctaatgctggacaacgaatgccaagggcacgatatggacttatacatgttgcaaacaataattatgacccatggagtatatatgctattggtgggagttcaaatccaaccattctaagtgaagggaatagtttcactgcaccaaatgatagcgacaagaaggaagtaacaagacgtgtagggtgtgaatcaccatcaacttgtgcaaactgggtgtggagatctacacaagattcttttaataatggagct

SEQ ID NO: 18 - DNA, 762 bp, Artificial Sequence
  Cha o 1 variant
gcccctggaaccctccgatacggagccacccgagaacgatcgctctggataattttcagcaagaacctcaatatcaagcttaatatgcctttatacattgccggaaacaagacgattgatggacgtggtgccgaagtgcatattggaaacggaggaccctgcctctttatgcgaaccgtctcgcatgtgattctccacggtttaaacatccacggatgcaacaccagcgtgtccggaaacgtgttaatctcggaggccagcggtgtcgtccctgtgcatgcccaagatggagacgctattaccatgcgaaacgtgaccgatgtgtggattgatcataacagcctttcggatagcagcgatggattagtagatgtgaccttagcctcgacgggtgtgaccatttcgaataaccattttttcaatcatcataaagtcatgttattaggacatagcgatatctatagcgatgacaaaagcatgaaggtcaccgtcgccttcaaccagtttggtcctaacgccggtcagcgaatgcctcgagcccgatatggtttaatccatgtggccaataacaactatgacccttggagcatctatgccattggaggaagcagcaaccctactattctcagcgaaggaaacagcttcaccgcccctaacgattcggacaagaaggaagtaacccgtcgagtaggatgcgaaagccctagcacctgcgccaattgggtctggcgtagcacccaggatagctttaacaacggtgcc

SEQ ID NO: 19 - protein, 372 aa, Artificial Sequence
  Amb a 1 variant
AEGVGEILPSVNETRSLQACEALNIIDKCWRGKADWENNRQALADCAQGFAKGTYGGKWGDVYTVTSNLDDDVANPKEGTLRFAAAQNRPLWIIFKNDMVINLNQELVVNSDKTIDGRGVKVEIINGGLTLMNVKNIIIHNINIHDVKVLPGGMIKSNDGPPILRQASDGDTINVAGSSQIWIDHCSLSKSFDGLVDVTLGSTHVTISNCKFTQQSKAILLGADDTHVQDKGMLATVAFNMFTDNVDQRMPRCRFGFFQVVNNNYDRWGTYAIGGSSAPTILCQGNRFLAPDDQIKKNVLARTGTGAAESMAWNWRSDKDLLENGAIFVTSGSDPVLTPVQSAGMIPAEPGEAAIKLTSSAGVFSCHPGAPC

SEQ ID NO: 20 - protein, 353 aa, Artificial Sequence
  Cry j 1 variant
DNPIDSCWRGDSNWAQNRMKLADCAVGFGSSTMGGKGGDLYTVTNSDDDPVNPAPGTLRYGATRDRPLWIIFSGNMNIKLKMPMYIAGYKTFDGRGAQVYIGNGGPCVFIKRVSNVIIHGLHLYGCSTSVLGNVLINESFGVEPVHPQDGDALTLRTATNIWIDHNSFSNSSDGLVDVTLSSTGVTISNNLFFNHHKVMLLGHDDAYSDDKSMKVTVAFNQFGPNCGQRMPRARYGLVHVANNNYDPWTIYAIGGSSNPTILSEGNSFTAPNESYKKQVTIRIGCKTSSSCSNWVWQSTQDVFYNGAYFVSSGKYEGGNIYTKKEAFNVENGNATPQLTKNAGVLTCSLSKRC

SEQ ID NO: 21 - protein, 346 aa, Artificial Sequence
  Cup a 1 variant
DNPIDSCWRGDSNWDQNRMKLADCVVGFGSSTMGGKGGEIYTVTSSEDNPVNPTPGTLRYGATREKALWIIFSQNMNIKLQMPLYVAGYKTIDGRGAVVHLGNGGPCLFMRKASHVILHGLHIHGCNTSVLGDVLVSESIGVEPVHAQDGDAITMRNVTNAWIDHNSLSDCSDGLIDVTLGSTGITISNNHFFNHHKVMLLGHDDTYDDDKSMKVTVAFNQFGPNAGQRMPRARYGLVHVANNNYDQWNIYAIGGSSNPTILSEGNSFTAPNESYKKEVTKRIGCETTSACANWVWRSTRDAFTNGAYFVSSGKAEDTNIYNSNEAFKVENGNAAPQLTQNAGVVA

SEQ ID NO: 22 - protein, 346 aa, Artificial Sequence
  Jun a 1 variant
DNPIDSCWRGDSNWDQNRMKLADCAVGFGSSTMGGKGGDFYTVTSTDDNPVNPTPGTLRYGATREKALWIIFSQNMNIKLKMPLYVAGHKTIDGRGADVHLGNGGPCLFMRKVSHVILHSLHIHGCNTSVLGDVLVSESIGVEPVHAQDGDAITMRNVTNAWIDHNSLSDCSDGLIDVTLGSTGITISNNHFFNHHKVMLLGHDDTYDDDKSMKVTVAFNQFGPNAGQRMPRARYGLVHVANNNYDPWNIYAIGGSSNPTILSEGNSFTAPSESYKKEVTKRIGCESPSACANWVWRSTRDAFINGAYFVSSGKTEETNIYNSNEAFKVENGNAAPQLTKNAGVVT

SEQ ID NO: 23 - protein, 354 aa, Artificial Sequence
  Cha o 1 variant
DNPIDSCWRGDANWDQNRMKLADCAVGFGSSAMGGKGGAFYTVTSSDDDPVNPAPGTLRYGATRERSLWIIFSKNLNIKLNMPLYIAGNKTIDGRGAEVHIGNGGPCLFMRTVSHVILHGLNIHGCNTSVSGNVLISEASGVVPVHAQDGDAITMRNVTDVWIDHNSLSDSSDGLVDVTLASTGVTISNNHFFNHHKVMLLGHSDIYSDDKSMKVTVAFNQFGPNAGQRMPRARYGLIHVANNNYDPWSIYAIGGSSNPTILSEGNSFTAPNDSDKKEVTRRVGCESPSTCANWVWRSTQDSFNNGAYFVSSGKNEGTNIYNNNEAFKVENGSAAPQLTKNAGVLTCILSKPCS

SEQ ID NO: 24 - protein, 376 aa, Artificial Sequence
  Art v 6 variant
ARADIGDELEAAQFNSTRRGLHECAAHNIIDKCWRCKADWEKNRQALAKCAQGFAKGTTGGLGGEIYVVTDCSDDNAANPKPGTLRCGVTQDKPLWIIFKKDMVIKLKHELVINKDKTIDGRGANVEITCGGLTIHNVCNVIIHNIHIHDIKVTEGGIIKATDAKPGHRHKSDGDGICVAGSSKIWIDHCTLSHGPDGLIDVTLGSTAVTISNCKFSHHQKILLLGADNSHVDDKKMHVTVAFNRFAEACDQRMPRCRFGFFQVVNNDYTSWGTYAIGGSANPTILSQGNRFHAPNDPMKKNVLVRADAPHTESMKWNWRSEKDLLENGAIFVASGCDPHLTPEQKSHLIPAEPGSAVLQLTSCAGTLKCVPGKPC

SEQ ID NO: 25 - DNA, 1119 bp, Artificial Sequence
  Amb a 1 variant
gccgaaggggtcggagaaatcttaccttcagttaacgaaacgaggagcctgcaagcatgtgaagcatacaacattatagacaagtgctggaggggcaaagccgattgggagaacaaccgacaagcgttagccgactgtgcccaaggttttgcaaagggaacctacggcggaaaatggggtgatgtctacacggtcaccagcaatctagatgatgatgttgcaaatccaaaagaaggcacactccggtttgctgccgcccaaaacaggcccttgtggatcatttttaaaaatgatatggtgattaatttgaatcaagagcttgtcgtaaacagcgacaagaccatcgatggccgaggagtgaaagttgaaatcattaacggaggtctcaccctcatgaatgtcaagaatataatcattcataacataaatatccatgatgttaaagtgcttccaggaggcatgattaagtccaacgatggtccaccaattttaagacaagcaagtgatggtgatactataaatgttgctggtagttcccaaatatggatagaccattgctcactcagcaagtctttcgatgggctggtcgatgtcaccctcggtagcacacacgtgaccatttccaactgcaaattcacccaacagtcaaaagcaatattgttgggggcagatgacacccatgttcaagataaaggaatgctagccacggtcgctttcaacatgttcaccgataacgttgaccaaagaatgcctagatgtcgatttgggtttttccaagttgttaacaacaactacgacagatggggaacgtacgccataggtggtagctcggccccaactatactctgccaagggaacagattcttggcccctgatgatcagatcaagaaaaatgtcctagcgaggactggtacaggcgctgctgagtcgatggcgtggaactggagatctgataaagacttgcttgaaaatggtgctatttttgttacatctgggtctgatccagtgctaacccctgttcaaagcgcagggatgattccagctgaaccaggagaagccgctataaaactcactagtagtgctggtgtactctcatgccgtcctggagcaccttgctaa

SEQ ID NO: 26 - DNA, 1119 bp, Artificial Sequence
  Amb a 1 variant
gccgaaggtgttggtgaaattttaccttcggtgaacgaaactcgtagcctacaggcatgcgaagcatacaacattatcgacaaatgctggcgtggtaaagccgattgggagaacaaccgacaggctttagccgactgcgcccagggctttgcaaaaggtacctacggtggtaaatggggcgatgtttacacggttaccagcaacctcgatgatgatgtggcaaacccgaaagaaggtacgttacggtttgccgccgcccagaaccgtcccttatggattatttttaaaaacgatatggttattaacttaaaccaggagcttgttgtaaacagcgacaaaaccattgatggtcgaggtgttaaagtggaaattattaacggtggcttaaccttaatgaacgttaaaaacatcattattcataacatcaacattcatgatgtgaaagttcttccgggtggtatgattaaatcgaacgatggcccgccgattttacgtcaggcaagcgatggcgatacgatcaacgtggccggcagctcgcagatctggatcgaccattgctcgttaagcaaatcgtttgatggtctagtggatgtgaccttaggcagcacgcacgttaccatttcgaactgcaaatttacccagcaatcgaaagcaatcttattaggtgcagatgacacccatgtgcaggataaaggtatgctcgccacggttgcctttaacatgtttaccgataacgtggaccagcgtatgcctcgttgccgatttggtttttttcaggtggtgaacaacaactacgaccgttggggtacttacgccatcggcggcagctcggccccgacgatcttatgccagggtaaccgttttttagcccctgatgatcaaattaaaaaaaacgttctcgctcgtacgggcacgggtgccgccgagtcgatggcttggaactggcgttcggataaagacttacttgaaaacggcgccatttttgtgacgtcgggttcggatccggttctcacccctgtgcagagcgcaggtatgattccggccgaaccgggtgaagccgccatcaaattaacgagcagcgccggcgtattatcgtgccgacctggtgcaccttgctaa

SEQ ID NO: 27 - DNA, 1062 bp, Artificial Sequence
  Cry j 1 variant
gataatcccatagacagctgctggagaggagactcaaactgggcacaaaacagaatgaagctcgcagattgtgcagtgggcttcggaagctccaccatgggaggcaagggaggagatctttatacggtcacgaactcagatgacgaccctgtgaatcctgcaccaggaactctgcgctatggagcaacccgagataggcccctgtggataattttcagtgggaatatgaatataaagctcaaaatgcctatgtacattgctgggtataagacttttgatggcaggggagcacaagtttatattggcaatggcggtccctgtgtgtttatcaagagagttagcaatgttatcatacacggtttgtatctgtacggctgtagtactagtgttttggggaatgttttgataaacgagagttttggggtggagcctgttcatcctcaggatggcgatgctcttactctgcgcactgctacaaatatttggattgatcataattctttctccaattcttctgatggtctggtcgatgtcactcttacttcgactggagttactatttcaaacaatctttttttcaaccatcataaagtgatgttgttagggcatgatgatgcatatagtgatgacaaatccatgaaggtgacagtggcgttcaatcaatttggacctaactgtggacaaagaatgcccagggcacgatatggacttgtacatgttgcaaacaataattatgacccatggactatatatgcaattggtgggagttcaaatccaaccattctaagtgaagggaatagtttcactgcaccaaatgagagctacaagaagcaagtaaccatacgtattggatgcaaaacatcatcatcttgttcaaattgggtgtggcaatctacacaagatgttttttataatggagcttattttgtatcatcagggaaatatgaagggggtaatatatacacaaagaaagaagctttcaatgttgagaatgggaatgcaactcctcaattgacaaaaaatgctggggttttaacatgctctctctctaaacgttgttga

SEQ ID NO: 28 - DNA, 1062 bp, Artificial Sequence
  Cry j 1 variant
gataaccctatcgacagttgctggcgtggcgactcgaattgggcccagaatcgtatgaaattagccgattgcgccgtgggttttggcagttcgacgatgggcggtaaaggcggcgatctgtatacagttacaaattcggatgacgacccggtgaacccggccccgggcaccctgcggtatggcgccacccgagataggcctctgtggatcatttttagcggtaacatgaacatcaaattaaaaatgccgatgtacattgccggttataaaacctttgatggtaggggcgcccaggtgtatattggtaacggtggtccttgcgtgtttatcaaacgtgtgagtaacgtgatcatccacggtctgtatctgtacggttgcagcaccagcgtgctgggtaacgtgctgatcaatgaaagctttggtgtggaaccggtgcatccgcaggatggtgatgccctgaccctgcggaccgccaccaacatttggattgatcataactcgttttcgaactcgtcggatggtctggttgatgttaccctgacctcgaccggcgtgaccatttcgaataacctgttttttaatcatcataaagtgatgctgttgggtcatgatgatgcctatagcgatgacaaatcgatgaaagtgaccgtggcttttaaccagtttggcccgaattgcggccagcgtatgcctagggcccgatatggcctggttcatgtggccaataacaactatgacccgtggaccatctatgccattggtggtagctcgaacccgaccattctcagcgaaggtaacagctttaccgccccgaacgaaagttacaaaaaacaggttaccatccgaattggctgcaaaacctcgtcgtcgtgctcgaactgggtgtggcagtcgacccaggatgtgttttataacggcgcctattttgtttcgtcgggtaaatatgaaggtggtaacatctacaccaaaaaagaagcctttaacgtggaaaacggtaacgccaccccgcagctgaccaaaaacgccggtgtgttgacctgctcgttatcgaaacgatgctga

SEQ ID NO: 29 - DNA, 1041 bp, Artificial Sequence
  Cup a 1 variant
gataatcccatcgacagctgctggagaggagattcgaactgggatcaaaacagaatgaagctcgcagactgtgttgtgggatttggaagctcgaccatgggaggcaaaggaggagaaatttacaccgtcacaagctcagaagataatcctgtgaatcctacaccaggaactttgcgctatggagcaacaagagaaaaagcactttggataattttctctcagaatatgaatataaagctccagatgcctttgtatgttgctggatataagactattgacggcaggggagcagttgttcatcttggcaatggcggtccctgtctgtttatgaggaaagcgagccatgttattctccatggtttgcatatacacggttgtaatacgagtgttttgggggatgttttggtaagtgagtccattggtgtggagcctgttcatgctcaggatggggacgccattactatgcggaatgttacaaatgcttggattgatcataattctctctccgattgttctgatggtcttatcgatgttacacttggttccactggaattactatctccaacaatcacttcttcaaccatcataaagtgatgttgttaggacatgatgatacatatgatgatgacaaatctatgaaagtgacagtggccttcaatcaatttggacccaatgctgggcaaagaatgccaagggcacgatatggacttgtacatgttgcaaacaataattatgatcaatggaatatatatgctattggtgggagttcaaatccaaccattctaagtgaagggaatagtttcactgctccaaatgagagctacaaaaaggaagtaacaaagcgtatagggtgtgaaacaacatcagcttgtgcgaactgggtgtggagatccacacgagatgcttttactaatggagcttattttgtatcatcggggaaagctgaagacaccaatatatacaatagtaatgaagctttcaaagttgagaatgggaatgcagctcctcaattaacacaaaatgctggagttgtagcataa

SEQ ID NO: 30 - DNA, 1041 bp, Artificial Sequence
  Cup a 1 variant
gataaccccatcgacagctgctggcgaggcgattcaaattgggatcaaaatcgaatgaaactcgcagactgcgtggtgggcttcggcagctcaacgatgggcggaaaaggcggcgaaatttacacggtaaccagctcggaagataaccctgtgaaccctacccctggcaccttaaggtatggcgcaacccgagaaaaagcattatggatcattttttcgcaaaacatgaacatcaaactccaaatgcctttatatgtggccggctataaaaccattgacggacgaggcgcagtggtgcatttaggaaacggaggtccctgcctattcatgcgaaaagctagccatgtgattctccacggtttacatatccacggttgcaacacaagcgtgttaggtgatgtgttagtaagcgaatcgattggtgtggaacctgtgcatgcccaagatggtgacgctattaccatgcgaaacgtgaccaacgcctggattgatcataactcgctctcggattgctcggatggtttaatcgatgtgaccttaggttcgaccggcattaccatctcgaataaccacttttttaatcatcataaagtgatgttactaggccatgatgatacctatgatgatgacaaatcgatgaaagtgaccgtggcttttaaccaattcggccccaacgccggtcaacgaatgcctcgggcacgatatggcttagtacatgtggcaaataacaactatgatcaatggaacatctatgccattggtggtagctcgaaccctacgattctaagcgaaggtaacagctttaccgcccctaacgaaagctacaaaaaagaagtaaccaaaaggatcggttgcgaaaccacctcggcctgcgctaattgggtgtggcgatcgacccgagatgccttcaccaacggcgcctatttcgtatcgtcaggtaaagccgaagacacgaatatctacaatagcaatgaagcctttaaagtggaaaatggtaatgcagcccctcaactaacccaaaatgccggcgtggtagcataa

SEQ ID NO: 31 - DNA, 1131 bp, Artificial Sequence
  Jun a 1 variant
gctcgggctgacattggtgatgagctcgaagcggctcaatttaattcaacaaggaggggcttacacgaatgtgcagcacataacataatagacaagtgttggaggtgcaaagctgattgggaaaaaaaccgacaagcattagccaaatgcgcgcaaggttttgcaaagggaacaactggcggattgggaggggaaatctacgtggtgactgattgttcagatgacaatgctgcaaatccaaagccagggacacttcgttgtggtgtcacccaagataaacctttgtggatcatctttaagaaagatatggtcataaaacttaaacacgagcttgtgataaacaaagacaagacaattgatggaagaggtgcaaatgttgagatcacttgtggcggtctcaccattcacaacgtttgcaatgtgatcattcataacattcacatacatgatattaaagtaaccgaaggtggaattattaaggcaacggacgctaaaccaggacatagacataagagcgacggagatggtatttgtgttgctggttcttcaaagatatggatcgatcattgcacacttagtcatggtccagatggccttattgatgtcacgttgggtagcacagccgttaccatttccaattgcaaatttagccatcaccaaaaaattctattactcggagcagacaattcacatgtagacgataaaaaaatgcatgtcacagtagcattcaacaggttcgcagaagcatgtgatcaaagaatgccacgatgtcgatttggatttttccaagttgttaacaatgactacaccagctggggaacgtacgccattggtggtagtgccaatcctactatccttagccaaggcaaccgattccatgctccgaatgacccaatgaagaaaaatgtgttggtgagggctgatgcaccacatacagagtcaatgaagtggaattggagatctgagaaagacttgttagaaaatggagctatatttgtagcatcagggtgcgacccgcatctaaccccggaacaaaaaagccatttgattccagctgaaccaggatcagcagttcttcaactcaccagttgtgctggcacgctcaaatgcgttcctggaaaaccttgttaa

SEQ ID NO: 32 - DNA, 1131 bp, Artificial Sequence
  Jun a 1 variant
gcgagggcggacattggcgatgagcttgaagctgcgcaatttaattcgacgcgacgaggtctccacgaatgcgccgcccataatatcatcgacaaatgctggcgatgcaaagcggattgggaaaaaaatcgacaagccctcgcaaaatgcgctcaaggctttgccaaaggcacgacgggtggcttaggcggagaaatctacgtagtaacggattgctcggatgacaatgcggccaatcctaaacctggaacgttacggtgcggcgtcacccaagataaaccgttatggatcatctttaaaaaagatatggtcatcaaattaaaacacgagttagtaatcaataaagacaaaacgattgatggccgaggcgccaatgtggagatcacgtgcggtggccttaccattcacaatgtgtgcaatgtaatcattcataatattcacatccatgatattaaagtaaccgaaggcggcattattaaagccactgacgcgaaacctggccatcgacataaaagtgacggcgatggcatttgcgtggcgggctcgtcgaaaatctggatcgatcattgcacgttaagtcatggccctgatggtttaattgatgtcactttaggcagtacggcagtgaccatttccaattgcaaatttagtcatcaccaaaaaattctactacttggcgccgacaattcgcatgtagacgataaaaaaatgcatgtcacggtagccttcaatcgattcgccgaagcctgcgatcaacgaatgcctcgatgccgatttggctttttccaagtggtgaataatgactacaccagttggggcacttacgcaattggcggcagtgcaaatccgacgatcttaagtcaaggtaatcgattccatgcgcccaatgaccctatgaaaaaaaatgtattagtacgagcggatgcccctcatacggagtcgatgaaatggaattggcgatcggagaaagacttactcgaaaatggcgcgatctttgtagcctcgggatgcgacccccatctaacccccgaacaaaaaagtcatttaattcctgcggaacctggctcggccgtgttacaacttaccagttgcgcgggtactcttaaatgcgtgccgggcaaaccgtgctaa

SEQ ID NO: 33 - DNA, 1041 bp, Artificial Sequence
  Cha o 1 variant
gataatcccatcgacagctgctggagaggagattcgaactgggatcaaaacagaatgaagctcgcagactgtgctgtgggatttggaagctccaccatgggaggcaaaggaggagatttttacaccgtcacaagcacagatgataatcctgtgaatcctacaccaggaactttgcgctatggagcaacaagagaaaaagcactttggatcattttctctcagaatatgaatataaagctcaagatgcctttgtatgttgctggacataagactattgacggcaggggagcagatgttcatcttggcaacggcggtccctgtctgtttatgaggaaagtgagccatgttattctccatagtttgcatatacacggttgtaatacgagtgttttgggggatgttttggtaagtgagtctattggggtggagcctgttcatgctcaggatggggacgccattactatgcgcaatgttacaaatgcttggattgatcataattctctctccgattgttctgatggtcttatcgatgttacgcttggctccactggaattactatctccaacaatcacttcttcaaccatcataaagtgatgttgttaggacatgatgatacatatgacgatgacaaatctatgaaagtgacagtggcgttcaatcaatttggacctaatgctgggcaaagaatgccaagggcacgatatggacttgtacatgtcgcaaacaataattatgatccatggaatatatatgctattggtgggagttcaaatccaaccattctaagtgaagggaatagtttcactgccccaagtgagagctacaagaaggaagtaacaaagcgtatagggtgtgaatcaccatcagcttgtgcgaactgggtgtggagatctacacgagatgcttttattaatggagcttattttgtatcatcggggaaaactgaagagaccaatatatacaatagtaatgaagctttcaaagttgagaatgggaatgcagctcctcaattaaccaaaaatgctggagttgtaacctaa

SEQ ID NO: 34 - DNA, 1041 bp, Artificial Sequence
  Cha o 1 variant
gataaccccatcgacagctgctggcgaggcgatagtaattgggatcaaaatcgaatgaaactcgccgactgcgccgttggctttggcagctcgacgatgggcggaaaaggcggcgatttttacacggtcaccagcaccgatgataacccggttaacccgaccccgggcaccttaaggtacggcgccacccgagagaaagccttatggatcatttttagccaaaacatgaacatcaaactcaaaatgccgttatacgtggccggccataaaaccattgacggacgaggcgccgatgtgcatttaggaaatggaggaccctgcctatttatgcgaaaagttagccatgtgattctccatagcttacatatccacggatgcaacacgagcgtgttaggtgatgtgttagttagcgagagcattggtgttgagccggtgcatgcccaagatggtgacgctattaccatgaggaacgtgaccaacgcctggattgatcataacagcctctcggattgcagcgatggattaatcgatgtgacgttaggatcgaccggcattaccatctcgaataaccacttttttaatcatcataaagttatgttactcggccatgatgatacctacgacgatgacaaaagcatgaaagttaccgttgcttttaaccaatttggcccgaacgccggtcaacgaatgccgcgagcccgatacggcttagttcatgtagccaataacaactacgatccgtggaacatctacgccattggaggtagctcgaacccgacgattctaagcgagggtaacagctttaccgctccgagcgagagctacaaaaaagaggttaccaaacggatcggttgcgagtcgccgtcggcctgcgctaattgggtttggcgatcgacccgagatgcctttattaacggcgcctactttgtttcgtcaggtaaaaccgaggagacgaacatctacaacagcaacgaggcctttaaagtggagaacggtaacgccgccccgcaactcacgaaaaacgccggcgtggttacgtga

SEQ ID NO: 35 - DNA, 1065 bp, Artificial Sequence
  Art v 6 variant
gataatcccattgacagctgctggagaggagatgcaaactgggatcaaaacaggatgaagctcgcagactgtgcagtgggcttcggaagctccgccatgggaggcaaaggaggagctttttacactgtcacaagctcagatgacgaccctgtaaatcctgcaccaggtactctgcgctacggggcaacacgagaaaggtcactgtggatcattttctctaagaatctgaacataaagctcaacatgcctttgtacattgctgggaataagaccattgatggcagaggagcagaagttcatattggcaatggtggtccctgtctgtttatgaggacagtgagccatgttattctacacggattgaatatacacggctgtaatacaagtgtttcggggaatgttttgataagcgaggcttctggagtggtgcctgttcatgctcaggatggcgacgccattactatgcgcaatgttacagatgtttggattgatcataattctctctccgattcttctgatggtcttgtcgatgttacacttgcttccaccggagttactatttccaacaatcattttttcaaccatcataaagtgatgttgttagggcatagtgatatatatagtgatgacaaaagtatgaaggtgacagtggcattcaatcaatttggacctaatgctggacaacgaatgccaagggcacgatatggacttatacatgttgcaaacaataattatgacccatggagtatatatgctattggtgggagttcaaatccaaccattctaagtgaagggaatagtttcactgcaccaaatgatagcgacaagaaggaagtaacaagacgtgtagggtgtgaatcaccatcaacttgtgcaaactgggtgtggagatctacacaagattcttttaataatggagcttattttgtatcttcagggaaaaatgaagggactaatatatacaacaataatgaagctttcaaagttgagaatgggagtgcagctcctcaattaacaaaaaatgctggggttctaacatgcattctctctaaaccttgctcatga

SEQ ID NO: 36 - DNA, 1065 bp, Artificial Sequence
  Art v 6 variant
gataaccccattgactcgtgttggcgtggtgatgccaattgggatcagaatcgaatgaagcttgccgactgcgccgtcggattcggttcgtcggctatgggtggaaaaggtggtgccttttacaccgtaacctcgtcggatgacgaccctgtaaaccctgcccctggaaccctccgatacggagccacccgagaacgatcgctctggataattttcagcaagaacctcaatatcaagcttaatatgcctttatacattgccggaaacaagacgattgatggacgtggtgccgaagtgcatattggaaacggaggaccctgcctctttatgcgaaccgtctcgcatgtgattctccacggtttaaacatccacggatgcaacaccagcgtgtccggaaacgtgttaatctcggaggccagcggtgtcgtccctgtgcatgcccaagatggagacgctattaccatgcgaaacgtgaccgatgtgtggattgatcataacagcctttcggatagcagcgatggattagtagatgtgaccttagcctcgacgggtgtgaccatttcgaataaccattttttcaatcatcataaagtcatgttattaggacatagcgatatctatagcgatgacaaaagcatgaaggtcaccgtcgccttcaaccagtttggtcctaacgccggtcagcgaatgcctcgagcccgatatggtttaatccatgtggccaataacaactatgacccttggagcatctatgccattggaggaagcagcaaccctactattctcagcgaaggaaacagcttcaccgcccctaacgattcggacaagaaggaagtaacccgtcgagtaggatgcgaaagccctagcacctgcgccaattgggtctggcgtagcacccaggatagctttaacaacggtgcctattttgtaagcagcggaaaaaacgaaggaaccaacatctacaataacaacgaagccttcaaagtggagaacggaagcgccgcccctcagttaaccaaaaacgccggagtgctcacctgtattcttagcaaaccttgtagctga

SEQ ID NO: 37 - protein, 5 aa, Artificial Sequence
  enterokinase protease cleavage sites
DDDDK

SEQ ID NO: 38 - protein, 4 aa, Artificial Sequence
  factor Xa protease cleavage sites
IEGR

SEQ ID NO: 39 - protein, 4 aa, Artificial Sequence
  factor Xa protease cleavage sites
IDGR

SEQ ID NO: 40 - protein, 6 aa, Artificial Sequence
  thrombin protease cleavage sites
LVPRGS

SEQ ID NO: 41 - protein, 7 aa, Artificial Sequence
  TEV protease cleavage sites
ENLYFQG

SEQ ID NO: 42 - protein, 8 aa, Artificial Sequence
  Precission protease cleavage sites
LEVLFQGP

SEQ ID NO: 43 - protein, 13 aa, Artificial Sequence
  Cry j 1 fragment
DNPIDSCWRGDSN

SEQ ID NO: 44 - protein, 10 aa, Artificial Sequence
  Cry j 1 fragment variant
GADNPIDSCW

SEQ ID NO: 45 - protein, 7 aa, Artificial Sequence
  Cry j 1 fragment
GPIDSCW

SEQ ID NO: 46 - protein, 8 aa, Artificial Sequence
  Cry j 1 fragment
GTPIDSCW

SEQ ID NO: 47 - protein, 8 aa, Artificial Sequence
  Cry j 1 fragment
GMPIDSCW

SEQ ID NO: 48 - protein, 21 aa, Artificial Sequence
  pel B leader
KYLLPTAAAGLLLLAAQPAMA

SEQ ID NO: 49 - protein, 21 aa, Artificial Sequence
  pel C leader
KSLIPTITAGLLLALSQPLLA

(19) United States
(12) Patent Application Publication
(10) Pub. No.: US 2014/0242668 A1
(43) Pub. Date: Aug. 28, 2014

(54) POLYSACCHARIDE LYASES

(71) Applicants: Michael Wallner, Wals (AT); Michael Hauser, Zams (AT); Gabriele Gadermaier, Hallwang (AT); Martin Himly, Villach (AT); Fatima Ferreira, Salzburg (AT)

(72) Inventors: Michael Wallner, Wals (AT); Michael Hauser, Zams (AT); Gabriele Gadermaier, Hallwang (AT); Martin Himly, Villach (AT); Fatima Ferreira, Salzburg (AT)

(73) Assignee: BIOMAY AG, Vienna (AT)

(21) Appl. No.: 14/131,593

(22) PCT Filed: Jul. 9, 2012
(86) PCT No.: PCT/EP2012/063443

§ 371 Date: Jan. 8, 2014

(30) Foreign Application Priority Data
Jul. 8, 2011 (EP) 11173306.9

(51) Int. Cl. C12N 9/88

(52) U.S. Cl. CPC C12N 9/88
USPC 435/232; 536/23.2; 435/320.1

(57) ABSTRACT

The present invention relates to a variant of a mature polysaccharide lyase having a beta helix structure, wherein said variant comprises an N- and/or C-terminal truncation and consists essentially of at least 90% of the beta helix structure of the mature polysaccharide lyase.

The present invention relates to variants of polysaccharide lyases and methods for producing the same.

Plant polysaccharide lyases comprising a beta helix structure can hardly be expressed recombinantly in a host cell (see e.g. Takai T. Int Arch Allergy Immunol (2010);153(4):431-3 and Liu Z. et al. Int Arch Allergy Immunol (2010);153(4):347-58).

In order to enhance the recombinant expression rate of such proteins several strategies may be used. For instance, the codon usage of the nucleic acid molecules may be adapted to the host cells in which the lyases may be expressed. In an alternative approach it is also possible to identify those host cells which are able to expose the proteins of interest most efficiently. However, none of these strategies seem to overcome the problem of being able to recombinantly produce plant polysaccharide lyases, including allergens such as Amb a 1 and Cup a 1, having a beta helix structure.

Liu Z. et al. (Int Arch Allergy Immunol (2010);153(4):347-58) al constructed an N-terminal truncation of Jun a 1, including the signal peptide to end a peptide, which was predicted to mask the proteolytic site of Jun a 1. The truncated Jun a 1 was inserted into a viral vector and tobacco plants were transfected with the respective viral particles, however, the expression of truncated Jun a 1 in tobacco leafs was not successful.

In WO 2011/052697 Cry j 2, an allergenic polygalacturonase from Japanese cedar pollen, was produced as a vaccine candidate. The allergen was truncated and shuffled.

In WO 2007/140505 various fragments of Amb a 1 are disclosed for the possible usage as fusion fragments for generation of hypoallergens.

WO 2004/090099 relates to pectate layses and variants thereof. The variants of the pectate layses according to this international patent application may comprise truncations.

US 2009/304665 describes production of hyaluronan degrading enzymes and the GPI anchor responsible for anchoring in the membrane is removed by C-terminal truncations.

In WO 02/072833 fragments of the allergen Cup s 1 are disclosed.

It is an object of the present invention to provide methods and means for producing polysaccharide lyases which comprise a beta helix structure. A further object of the present invention is to provide hypoallergenic molecules which are derived from proteins, in particular polysaccharide lyases, comprising a beta helix structure and methods for producing them.

The present invention relates therefore to a variant of a mature polysaccharide lyase having a beta helix structure, wherein said variant comprises an N- and/or C-terminal truncation and consists of at least 90% of the beta helix structure of the mature polysaccharide lyase.

Plant polysaccharide lyases which comprise a beta helix structure are hardly expressed by recombinant methods due to their specific three-dimensional structure. Several attempts to recombinantly produce such lyases in various hosts failed. It turned now surprisingly out that such polysaccharide lyases can be recombinantly expressed when using variants thereof which essentially, i.e. of at least 90%, consist of the beta helix structure of the wildtype polysaccharide lyases.

Some of the polysaccharide lyases known in the art exhibit allergenic activity. For instance, some members of the pectate lyase family are known to be allergens. It turned out that variants of these pectate lyase family members do not exhibit or have a reduced allergenic activity compared to the wildtype pectate layses. Such variants are considered as hypoallergenic variants of these layses.

The hypoallergenic variant of a mature polysaccharide lyase having a beta helix structure of the present invention comprises an N- and/or C-terminal truncation, preferably an N- and a C-terminal truncation, and consists essentially of the beta helix structure of the mature polysaccharide lyase. As used herein, the term “variant consisting essentially of the beta helix structure” refers to a derivative/variant of a polysaccharide lyase which lacks essentially those parts of the wildtype polysaccharide lyase which are not associated to the beta helix structure. The phrase “consisting of” indicates that the listed elements are required or mandatory and that no other elements may be present. Thus, the phrases “consisting essentially of” and “consists of at least 90%” indicates that the listed elements are required or mandatory, but that other elements are optional and may or may not be present. The variant according to the present invention consisting “essentially of the beta helix structure” or “essentially only the beta helix structure” means that said variant is assembled to at least 90%, preferably at least 95%, more preferably at least 98%, of the beta helix structure of the wildtype polysaccharide lyase.

The phrase “polysaccharide lyase” as used herein refers to a group of enzymes that cleave polysaccharide chains via a beta-elimination mechanism resulting in the formation of a double bond at the newly formed non-reducing end (see e.g. Linhardt R J et al. Appl Biochem Biotechnol. 12 (1986):135-76).

“Mature polysaccharide lyase”, as used herein, refers to a polysaccharide lyase whose terminal signal sequence, if encoded by the respective cDNA, has been removed by maturation processes within the cell in which the polysaccharide lyase is naturally present.

“Beta helix structure” is a protein structure formed by the association of parallel beta strands in a helical pattern with either two or three faces. The structure is stabilized by interstrand hydrogen bonds and protein-protein interactions (see e.g. Yoder M D et al. FASEB J. 9 (1995):335-42). For identification of beta helix structures, a two step investigation can be performed using sequence as well as structure prediction software tools. In a first approach, the sequence of a given protein can be used for creation of a sequence homology alignment (e.g. via a protein blast like blastp at the NCBI). This allows the assignment of a given protein sequence to a known protein family and/or protein superfamily. In a further or alternative approach, a given protein sequence can be used for protein structure prediction (e.g. secondary structure prediction server, Dundee University, http://www.compbio.dundee.ac.uk/www-jpred/ Cole C. et al. Nucleic Acids Res (2008) 1; 36(Web Server Issue):W197-201) to confirm the blast alignment and to predict secondary structure elements to assign a given protein sequence to the polysaccharide lyase families, which contain a beta-helix core element, one or both of these approaches can be applied. According to this secondary structure prediction, the first beta strand of a consecutive cluster of at least eight beta strands is to be designated as the start of the beta helix, whereas the last beta strand of a consecutive cluster of at least eight, preferably 10, more preferably 12, even more preferably 14, beta strands of this prediction is to be assigned the endpoint of the hypothetical beta helix.

The “variants”, “proteins”, “polypeptides” and “peptides” referred to are “isolated” “variants”, “proteins”, “polypeptides” and “peptides”.

According to a preferred embodiment of the present invention the N- and/or C-terminal truncation comprises at least 10 amino acid residues.

As mentioned above the variant according to the present invention consists essentially of the beta helix structure of the wildtype polysaccharide lyase. Such variants comprise N- and/or C-terminal truncations of at least 10, preferably 11, 12, 13, 14, 15, 16, 17, 18, 19 or 20, more preferably 21, 22, 23, 24, 25, 26, 27, 28, 29 or 30, even more preferably 31, 32, 33, 34, 35, 36, 37, 38, 39, 40, 41, 42, 43, 44, 45, 46, 47, 48, 49 or 50, amino acid residues in respect to the wildtype polysaccharide lyase. Of course the number of truncated amino acid residues may vary between the N- and C-terminus of the polysaccharide lyase and may depend on the at which amino acid position within the polysaccharide lyase the beta helix structure begins or ends, respectively.

According to a particularly preferred embodiment of the present invention said N-terminal truncation comprises the region extending from the N-terminal end of the polysaccharide lyase to approximately 1 to 50, preferably 1 to 40, more preferably 1 to 30, even more preferably 1 to 20, particularly 1 to 10, amino acid residues before the N-terminus of the first beta sheet being part of the beta helix structure and said C-terminal truncation comprises the region extending from the C-terminal end of the polysaccharide lyase to approximately 1 to 50, preferably 1 to 40, more preferably 1 to 30, even more preferably 1 to 20, particularly 1 to 10, amino acid residues after the C-terminus of the last beta sheet part of the beta helix structure, under the provisio that the first and last beta sheet of the beta helix structure remains intact.

The variant according to the present invention may comprise an N-terminal truncation which includes the region of the polysaccharide lyase from the N-terminal end to the first amino acid residue being part of the first beta sheet being part of the beta helix structure. The C-terminal truncation may include the region of the polysaccharide lyase from the C-terminal end to the last amino acid residue of the last beta sheet being part of the beta helix structure. Consequently the N- and/or C-terminal truncation may involve 1 to 50, 1 to 49, 1 to 48, 1 to 47, 1 to 46, 1 to 45, 1 to 44, 1 to 43, 1 to 42, 1 to 41, 1 to 40, 1 to 39, 1 to 38, 1 to 37, 1 to 36, 1 to 35, 1 to 34, 1 to 33, 1 to 32, 1 to 31, 1 to 30, 1 to 29, 1 to 28, 1 to 27, 1 to 26, 1 to 25, 1 to 24, 1 to 23, 1 to 22, 1 to 21, 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3 or 1 to 2.

According to a preferred embodiment of the present invention the polysaccharide lyase is a member of a family of proteins selected from the group consisting of pectate lyase (EC 4.2.2.2), hyaluronate lyase (EC 4.2.2.1), poly(β-D-mannuronate) lyase (EC 4.2.2.3), chondroitin AC lyase (EC 4.2.2.5), oligogalacturonide lyase (EC 4.2.2.6), heparin lyase (EC 4.2.2.7), heparin-sulfate lyase (EC 4.2.2.8), pectate disaccharide-lyase (EC 4.2.2.9), pectin lyase (EC 4.2.2.10), poly(α-L-guluronate) lyase (EC 4.2.2.11); xanthan lyase (EC 4.2.2.12), exo-(1-->4)-α-D-glucan lyase (EC 4.2.2.13), glucuronan lyase (EC 4.2.2.14), anhydrosialidase (EC 4.2.2.15), levan fructotransferase (EC 4.2.2.16), inulin fructotransferase (EC 4.2.2.17, EC 4.2.2.18), chondroitin B lyase (EC 4.2.2.19), chondroitin-sulfate-ABC endolyase (EC 4.2.2.20), chondroitin-sulfate-ABC exolyase (EC 4.2.2.21) and pectate trisaccharide-lyase (EC 4.2.2.22).

As mentioned above some allergens are polysaccharide lyases, in particular pectate lyases. Therefore the variant according to the present invention being derived from a polysaccharide lyase is preferably an allergenic pectate lyase, preferably derived from Chamaecyparis obtusa (Japanese cypress), Cryptomeria japonica (Sugi), Cupressus arizonica (Cypress), Juniperus ashei (Mountain cedar), Artemisia vulgaris (Mugwort) and Ambrosia artemisiifolia (Short ragweed).

According to a preferred embodiment of the present invention the allergen is selected from the group consisting of Amb a 1, Art v 6, Cha o 1, Cup a 1, Cry j 1 and Jun a 1.

It turned surprisingly out that a variant of an allergen consisting of at least 90% of the beta helix structure as defined herein exhibits a reduced IgE reactivity compared to the wildtype allergen. Therefore the variant according to the present invention derived from an allergen is to be considered as a hypoallergenic molecule.

As used herein, the term “hypoallergenic” refers to the ability of a peptide, polypeptide or protein derived from an allergen with allergenic properties exhibiting reduced or no allergic reactions when administered to an individual. The reduced or missing ability of “hypoallergenic” derivatives of an allergen to induce an allergic reaction in an individual is obtained by removing or destroying the IgE binding epitopes from said allergens. According to a preferred embodiment of the present invention the hypoallergenic variant of a mature polysaccharide lyase having a beta helix structure exhibits an IgE reactivity which is at least 10%, preferably at least 20%, more preferably at least 30%, in particular at least 50%, lower than the IgE reactivity of the wild-type mature polysaccharide lyase.

Hypoallergenicity of polypeptides can be routinely tested by IgE western blots using pools of appropriate patients' sera. Reduction of IgE binding capacity of the respective hypoallergen will be assessed compared to the IgE binding capacity of the wild-type molecule. Also in the US 2009/0104208 (see e.g. example 2) methods for determining the IgE reactivity are described.

Variants of allergenic pectate lyases can be used as active pharmaceutical ingredients in medicaments for specific immunotherapy of allergies. Such medicaments can be prepared for the treatment of different allergies depending on the pectate lyase variant(s) contained therein. For example, a medicament comprising at least one variant of Amb a 1 can be for treatment of ragweed allergy, a medicament comprising at least one variant of Cry j 1 can be used for the treatment of Japanese cedar (Sugi) allergy, and a medicament comprising variants of Amb a 1 and Cry j 1 can be used for the treatment of both ragweed and Japanese cedar allergy. Therefore it is particularly preferred that the variant of the mature polysaccharide lyase of the present invention is Amb a 1 or Cry j 1.

Hypoallergenic variants of pectate lyases are particularly useful for specific immunotherapy.

A particularly preferred embodiment of the present invention relates therefore to a vaccine formulation for the treatment of an allergy comprising at least one hypoallergenic pectate lyase variant. Said formulation preferably further comprises at least one adjuvant, pharmaceutical acceptable excipient and/or preservative.

In order to increase the immunogenicity of the hypoallergenic molecules according to the present invention, adjuvants, for instance, may be used in a medicament according to the present invention. An adjuvant according to the present invention is an auxiliary agent which, when administered together or in parallel with an antigen, increases its immunogenicity and/or influences the quality of the immune response. Hence, the adjuvant can, e.g., considerably influence the extent of the humoral or cellular immune response. Customary adjuvants are, e.g., aluminum compounds, lipid-containing compounds or inactivated mycobacteria.

Generally, adjuvants can be of different forms, provided that they are suitable for administration to human beings. Further examples of such adjuvants are oil emulsions of mineral or vegetal origin, mineral compounds such as aluminium phosphate or hydroxide, or calcium phosphate, bacterial products and derivatives, such as P40 (derived from the cell wall of Corynebacterium granulosum), monophosphoryl lipid A (MPL, derivative of LPS) and muramyl peptide derivatives and conjugates thereof (derivatives from mycobacterium components), alum, incomplete Freund's adjuvant, liposyn, saponins (e.g. QS21), squalene, etc. (see, e.g., Gupta R. K. et al. (Vaccine 11:293-306 (1993)) and Johnson A. G. (Clin. Microbial. Rev. 7:277-289). The medicament of the present invention comprises most preferably alum as adjuvant.

The variant according to the present invention derived from Amb a 1 consists essentially of amino acid sequence SEQ ID No: 1, the variant derived from Cry j 1 consists essentially of amino acid sequence SEQ ID No: 2, the variant derived from Cup a 1 consists essentially of amino acid sequence SEQ ID No: 3, the variant derived from Jun a 1 consists essentially of amino acid sequence SEQ ID No: 4, the variant derived from Cha o 1 consists essentially of amino acid sequence SEQ ID No: 5 and the variant derived from Art v 6 consists essentially of amino acid sequence SEQ ID No: 6.

The pectate lyase variants, proteins and polypeptides of the present invention can be produced (manufactured) by recombinant expression in a host cell. The present invention is not restricted to a certain expression targeting (i.e. the locus of protein expression). Accordingly, the pectate lyase variants according to the invention may be expressed inside the host cell (intracellular, cytoplasmic or periplasmic) or outside the host cell (attached to the cell wall or outer membrane) or secreted into the culture medium (extracellular). If the pectate lyase variants are expressed intracellular, the proteins may be either soluble or insoluble as inclusion bodies.

Another aspect of the present invention relates to a polypeptide comprising a secretion signal sequence fused directly or via a linker to a variant according to the present invention as defined above, namely a pectate lyase, hyaluronate lyase, poly(β-D-mannuronate) lyase, chondroitin AC lyase, oligogalacturonide lyase, heparin lyase, heparin-sulfate lyase, pectate disaccharide-lyase, pectin lyase, poly(α-L-guluronate) lyase, xanthan lyase, exo-(1-->4)-α-D-glucan lyase, glucuronan lyase, anhydrosialidase, levan fructotransferase, inulin fructotransferase, chondroitin B lyase, chondroitin-sulfate-ABC endolyase, chondroitin-sulfate-ABC exolyase and pectate trisaccharide-lyase, preferably selected from the group consisting of Amb a 1, Art v 6, Cha o 1, Cup a 1, Cry j 1 and Jun a 1.

It turned surprisingly out that polysaccharide lyases and variants thereof as defined in the present invention show a higher stability and can be recombinantly expressed in higher yields. In order to avoid the removal of the secretion signal sequence within a host cell it is particularly advantageous to use heterologous secretion signal sequences and/or to introduce a linker between the secretion signal sequence and the variant or the mature polysaccharide lyase of the present invention which does not contain a naturally occurring protease cleavage site. In order to obtain a protein product without the secretion signal sequence it is advantageous to provide a linker which comprises an artificial protease cleavage site.

Secretion signal sequences or peptides are useful in directing the secretion of the polypeptides of the present invention. In certain embodiments, the polypeptide may comprise its own signal peptide, i.e., a “native” signal peptide. In other embodiments, the polypeptide may be fused to a “heterologous” secretion signal sequence/peptide, i.e., a signal peptide that is derived from a protein that is different from the wild-type polypeptide.

According to a preferred embodiment of the present invention the secretion signal sequence is a bacterial, fungal or eukaryotic, in particular mammalian, origin.

In a particularly preferred embodiment of the present invention the secretion signal sequences are bacterial signal peptides. Bacterial secretion signal sequences differ from each other only in minor aspects. For instance, signal sequences of gram-positive bacteria typically have a slightly longer and more basic N-terminus than signal peptides from other bacteria. Alternatively eukaryotic secretion signal sequences/peptides may be employed. For example, it has been shown that the signal peptides of E. coli periplasmic and outer membrane proteins, of vacuolar and secreted protein of Saccharomyces cerevisiae, and of constitutively secreted and storage granule proteins of multi-cellular eukaryotes can be exchanged without affecting the targeting of the mature protein. Indeed, in certain non-naturally occurring environments (e.g. expression of eukaryotic genes in bacterial cells), secretion signal sequences/peptides of some proteins have been shown to mediate efficient secretion. For instance, insulin and ovalbumin are secreted by E. coli upon expression of these genes, and yeast is able to efficiently secrete various secretory proteins derived from multicellular eukaryotes.

According to a preferred embodiment of the present invention the secretion signal sequence is selected from the group consisting of a PelB, a OmpA, a SW, an EX, a PhoA, a OmpF, a PhoE, a MalE, an OmpC, a LPP, a LamB, an OmpT, a LTB, a phosphatase (phol) and an invertase (Suc) secretion signal sequence and an α-mating factor prepro-sequence.

Secretion signal sequences/peptides may be derived from extracellular bacterial proteins. Examples of signal peptides or signal sequences that may be incorporated into the polypeptide of the present invention include Bacillus subtilis PgsA signal sequence, E. coli OmpA signal sequence, E. coli Ag43 signal sequence, B. licheniformis penicillinase signal sequence, E. coli lipoprotein (lpp) signal sequence, Bacillus cereus penicillinase III signal sequence, S. aureus penicillinase signal sequence, Neisseria IgA protease signal sequence, Serratia serine proteins signal sequence, Pseudomonas endoglucanase signal sequence, E. coli STA enterotoxin signal sequence, Klebsiella or E. coli cloacin (colicin) signal sequence, Proteus or Serratia Shla/HpmA hemolysin signal sequence, Klebsiella pullalanase signal sequence, Pseudomonas, Erwinia, or Xanthomonas exoenzyme signal sequences, Aeromonas aerolysin signal sequence, Vibrio CTX toxin signal sequence, Pseudomonas or Neisseria Type IV pillin signal sequence, E. coli P-type pilin signal sequence, E. coli Type-I pilin signal sequence, Yersinia OM proteins (YOPS) signal sequences, E. coli or Caulobacter flagellin signal sequences, Shigella or Salmonella Invasion protein signal sequences, E. coli, Proteus, or Morganella HlyA hemolysin signal sequences, Bordatella pertussis CyaA cyclolysin signal sequence, Erwinia, Serratia, or Proteus protease signal sequences, Rhizobium NodO signal sequence, E. coli ColV colicin signal sequence, among others known in the art.

These or other signal peptide sequences can be identified according to routine techniques in the art. For instance, PrediSi (Prediction of Signal peptides) represents one exemplary tool for predicting signal peptide sequences and their cleavage positions in both bacterial and eukaryotic amino acid sequences (see, e.g., Hiller et al, Nucleic Acids Research. 32:W375-W379, 2004). This software tool is especially useful for the analysis of large datasets in real time with high accuracy, and is based on a position weight matrix approach improved by a frequency correction that takes in to consideration the amino acid bias present in proteins. Also available is a Hidden Markov Model method for the prediction of lipoprotein signal peptides of Gram-positive bacteria, trained on a set of 67 experimentally verified lipoproteins (see, e.g., Bagos et al, J. Proteome Res. 7:5082-5093, 2008).

The polypeptides of the present invention is used to a secretion signal sequence may be prepared using standard techniques. For example, DNA sequences encoding the polypeptide components of a desired fusion may be assembled separately, and ligated into an appropriate expression vector. The 3′ end of the DNA sequence encoding one polypeptide component can be ligated, with or without a peptide linker, to the 5′ end of a DNA sequence encoding the second polypeptide component so that the reading frames of the sequences are in phase. This. permits translation into a single fusion protein that retains the biological activity of both component polypeptides.

According to a particularly preferred embodiment of the present invention peptide linker sequences may be employed to separate the variant of the present invention from the secretion signal sequence. Such peptide linker sequences may be incorporated into the fusion protein using standard techniques well known in the art. The peptide linker sequence incorporated between the variant of the present invention and the secretion signal sequence comprises advantageously a protease cleavage site. The presence of one or more protease cleavage sites allows to remove from the variant of the present invention the secretion signal sequence either by the expression host itself or by the addition of a respective protease after the isolation of the polypeptide of the present invention from the cultivation medium in which the polypeptide of the present invention is secreted in the course of its recombinant production.

According to a particularly preferred embodiment of the present invention the protease cleavage site is capable of being cleaved by thrombin, chymotrypsin, trypsin, pepsin, subtilisin, enterokinase, factor Xa, TEV protease and PreScission protease, whereby TEV protease is particularly preferred.

The sequence encoding a protease cleavage site can be included between the secretion signal or leader sequence or the affinity tag and the variant, protein or polypeptide of the present invention, thus permitting—as mentioned before—the removal of these sequences after incubation with a specific protease. Suitable protease cleavage sites for incorporation into the polypeptides of the invention include enterokinase (cleavage site Asp-Asp-Asp-Asp-Lys) (SEQ ID NO: 37), factor Xa (cleavage site Ile-Glu-Gly-Arg (SEQ ID NO: 38) or Ile-Asp-GIy-Arg (SEQ ID NO: 39)), thrombin (cleavage site Leu-Val-Pro-Arg-Gly-Ser; Arg-Gly) (SEQ ID NO: 40), TEV protease (cleavage site Glu-Asn-Leu-Tyr-Phe-Gln-Gly) (SEQ ID NO: 41), PreScission protease (cleavage site Leu-Glu-Val-Leu-Phe-Gln-Gly-Pro) (SEQ ID NO: 42), inteins (self-splicing intein sequences that can be activated, e.g., by adjustments to pH (Chong et al., Gene, 792 (1997):27-281)) and the like.

In an alternative embodiment of the present invention the cleavage site may be a chemical cleavage site including tryptophan residues cleaved by 3-bromo-3-methyl-2-(2-nitrophenylmercapto)-3H-indole; cysteine residues cleaved by 2-nitroso-5-thiocyano benzoic acid; the dipeptides -Asp-Pro- or -Asn-Gly- which can be cleaved by acid and hydroxylamine, respectively; and a methionine residue which is specifically cleaved by cyanogen bromide (CNBr).

The linker sequence may generally be from 1 to 50, preferably 1 to 40, more preferably 1 to 30, even more preferably 1 to 20, most preferably 1 to 10 amino acids in length. Linker sequences are typically not required when the fusion of the variant of the present invention to the secretion signal sequence results in a protease cleavage site.

The variants, proteins and polypeptides of the present invention can also be fused directly or via a linker (which may be a protease cleavage site) to a tag which is employed for purification or for detection. In a particular embodiment, the variant, protein or polypeptide of the present invention comprises the glutathione-5-transferase protein tag which provides the basis for rapid high-affinity purification of the polypeptide of the invention. Indeed, such a GST-fusion protein can then be purified from cells via its high affinity for glutathione. Agarose beads can be coupled to glutathione and such glutathione-agarose beads bind GST-proteins. Thus, in a particular embodiment of the invention, the variant, protein or polypeptide of the invention is bound to a solid support. In a preferred embodiment, if the polypeptide of the invention comprises a GST moiety, the polypeptide is coupled to a glutathione-modified support. In a particular case, the glutathione modified support is a glutathione-agarose bead.

Another aspect of the present invention relates to a nucleic acid molecule encoding a variant of the polysaccharide lyase or a polypeptide according to the present invention.

According to a preferred embodiment of the present invention to nucleic acid molecule comprises a nucleotide sequence encoding for a leader sequence.

The nucleotide sequence encoding for a leader sequence which is provided at the 5′-end of and in-frame with the nucleotide sequence encoding the variant of the present invention allows once transcribed and translated to direct the secretion of the translated polypeptide out from a host cell. The leader sequence may be of any origin provided that the host cell in which the polypeptide is expressed is capable to recognise the respective secretion signal sequences.

The leader sequence is preferably selected from the group consisting of PelB and PelC.

The nucleic acid molecule according to the present invention can of course comprise further nucleotide sequences encoding for polypeptides and peptides which may facilitate the purification, enrichment or stabilisation of the variant encoded.

According to particular preferred embodiment of the present invention the nucleotide sequence of said nucleic acid molecule is codon optimised and/or codon harmonised.

Optimizing codons to achieve high levels of protein expression in host cells is an often used method. Such methods regularly comprise the identification of “non-preferred” or a “less preferred” codon and the replacement of one or more of these non-preferred or less preferred codons with a “preferred codon” encoding the same amino acid as the replaced codon and at least one non-preferred or less preferred codon in the nucleic acid has been replaced by a preferred codon encoding the same amino acid. A preferred codon is a codon over-represented in Boding sequences in genes in the host cell and a non-preferred or less preferred codon is a codon under-represented in coding sequences in genes in the host cell.

In order to stabilise the variants of the present invention within the cell the N-terminal amino acid residues, preferably the first two residues or at least the second, are preferably exchanged by respective stabilising amino acid residues (e.g. glycine) (see e.g. Varshaysky A. Nature Structural & Molecular Biology 15 (2008):1238-40; Dougan D A et al. Molecular Microbiology 76 (2010): 545-558; Tobias J W, et al. Science 254 (1991):1374-7).

Host cells for expressing the nucleic acids, expression cassettes and vectors of the invention include bacteria, yeast, fungi, plant cells, insect cells and mammalian cells. Thus, the invention provides methods for optimizing codon usage in all of these cells, codon-altered nucleic acids and polypeptides made by the codon-altered nucleic acids. Exemplary host cells include gram negative bacteria, such as Escherichia coli and Pseudomonas fluorescens; gram positive bacteria, such as Lactobacillus gasseri, Lactococcus lactis, Lactococcus cremoris, Bacillus subtilis. Exemplary host cells also include eukaryotic organisms, e.g., various yeast, such as Sacchammyces Sp., including Saccharomyces cerevisiae, Schizosaccharomyces pombe, Pichia pastoris and Kluyveromyces lactis, Hansenula polymorpha, Aspergillus niger, and mammalian cells and cell lines and insect cell lines. In a particularly preferred embodiment, Escherichia coli is the host cell. In another particularly preferred embodiment, Pichia pastoris is the host cell.

The invention also includes nucleic acids and polypeptides optimized for expression in these organisms and species. It is particularly preferred to use methods in which the codons are harmonised. Methods for harmonising codons are well known in the art (Angov E, et al. PloSOne 3 (2008):e2189). In such methods the codon usage of the host cell as well as of the cell from which the nucleic acid molecule encoding the polypeptide to be transcribed and translated is derived from is considered. This allows to obtain a nucleotide sequence whose codon usage is balanced between the host cell and the cell originating the nucleic acid molecule encoding the polypeptide to be expressed.

The codon usage for a given organism is available at the codon usage database http://www.kazusa.or.jp/codon/ (Nakamura Y. et al. Nucleic Acids Res (2000);28(1):292). The codon usage of pectate lyase genes was determined for the respective source organism. The codon usage of the host organism was used to transfer the codon usage for the gene of interest from the source organism to the host organism. Therefore, E. coli codons, for instance, encoding the respective amino acid at a given position and showing the codon usage, which was closest to the original codon of the source organism for the respective amino acid, were selected. The DNA code was then changed accordingly and the resulting gene with new codon composition was generated synthetically.

According to a preferred embodiment of the present invention the nucleic acid molecule encoding the variant of Amb a 1 has the nucleotide sequence SEQ ID No: 7 or SEQ ID No: 8, the nucleic acid molecule encoding Cry j 1 has the nucleotide sequence SEQ ID No: 9 or 10, the nucleic acid molecule encoding Cup a 1 has the nucleotide sequence SEQ ID No: 11 or 12, the nucleic acid molecule encoding Art v 6 has the nucleotide sequence SEQ ID No: 13 or 14, the nucleic acid molecule encoding Jun a 1 has the nucleotide sequence SEQ ID No: 15 or 16, and the nucleic acid molecule encoding Cha o 1 has the nucleotide sequence SEQ ID No: 17 or 18.

Another aspect of the present invention relates to a nucleic acid molecule having or comprising nucleic acid sequence SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 25, SEQ ID No. 26, SEQ ID No. 27, SEQ ID No. 28, SEQ ID No. 29, SEQ ID No. 30, SEQ ID No. 31, SEQ ID No. 32, SEQ ID No. 33, SEQ ID No. 34, SEQ ID No. 35 or SEQ ID No. 36. These nucleic acid molecules may be fused to nucleic acid sequences encoding a secretion signal sequence as described herein. Also encompassed by the present invention are the polypeptides encoded by these nucleic acid molecules.

Another aspect of the present invention relates to vector comprising a nucleic acid molecule according to the present invention.

The nucleic acid molecule of the present invention encoding the variant of the present invention may be operably linked to transcriptional and translational control sequences, e.g. promoters or enhancers, to direct or modulate RNA synthesis and expression. The expression control sequence can be in an expression vector. Exemplary bacterial promoters include lad, lacZ, T3, T7, gpt, lambda PR, PL, tac and trp. Exemplary yeast promoters include AOX1, GAP/GAPDH, ADH2, PGK, FLD1, MOX. Exemplary eukaryotic promoters include CMV immediate early, HSV thymidine kinase, early and late SV40, LTRs from retrovirus, and mouse metallothionein I.

Promoters suitable for expressing, or over-expressing, a polypeptide in bacteria include the E. coli lac or trp promoters, the lad promoter, the lacZ promoter, the T3 promoter, the T7 promoter, the gpt promoter, the lambda PR promoter, the lambda PL promoter, promoters from operons encoding glycolytic enzymes such as 3-phosphoglycerate kinase (PGK), and the acid phosphatase promoter. Eukaryotic promoters include the CMV immediate early promoter, the HSV thymidine kinase promoter, heat shock promoters, the early and late SV40 promoter, LTRs from retroviruses, and the mouse metallothionein-I promoter. Other promoters known to control expression of genes in prokaryotic or eukaryotic cells or their viruses may also be used. The present invention relates also to expression systems such as expression cassettes, vectors, cloning vehicles and the like, comprising nucleic acids of the invention, e.g., sequences encoding the variants of the invention, for expression, and over-expression, of the polypeptides of the invention. Expression vectors and cloning vehicles of the invention can comprise viral particles, baculovirus, phage, plasmids, phagemids, cosmids, fosmids, bacterial artificial chromosomes, viral DNA (e.g., vaccinia, adenovirus, foul pox virus, pseudorabies and derivatives of SV40), Pl-based artificial chromosomes, yeast plasmids, yeast artificial chromosomes, and any other vectors specific for specific hosts of interest (such as bacillus, Aspergillus and yeast). Exemplary bacterial vectors include pQE vectors (Qiagen), pBluescript plasmids, pNH vectors, (lambda-ZAP vectors (Stratagene); ptrc99a, pKK223-3, pDR540, pRIT2T (Pharmacia); Eukaryotic: pXTl, pSG5 (Stratagene), pSVK3, pBPV, pMSG, pSVLSV40 (Pharmacia), pVL1392, pVL1393, pAcSG2, pAcGP67, pAcUW21, pAcUW51, pAcMP2 and pAcMP3, and pAcGHLT (GST-fusion vectors) and pAcHLT (6×His-fusion vectors) [BD]. However, any other plasmid or other vector may be used so long as they are replicable and viable in the host. Low copy number or high copy number vectors may be employed with the present invention.

As representative examples of expression vectors which may be used there may be mentioned viral particles, baculovirus phage, plasmids, phagemids, cosmids, fosmids, bacterial artificial chromosomes, viral DNA (e.g., vaccinia, adenovirus, foul pox virus, pseudorabies and derivatives of SV40), Pl-based artificial chromosomes, yeast plasmids, yeast artificial chromosomes, and any other vectors specific for specific hosts of interest (such as bacillus, Aspergillus and yeast). Thus, for example, the DNA may be included in any one of a variety of expression vectors for expressing a polypeptide. Such vectors include chromosomal, nonchromosomal and synthetic DNA sequences. Large numbers of suitable vectors are known to those of skill in the art.

The nucleic acid sequence in the expression vector is operatively linked to an appropriate expression control sequence(s) (promoter) to direct RNA synthesis. Particular named bacterial promoters include lad, lacZ, T3, T7, gpt, lambda PR, PL and trp. Eukaryotic promoters include CMV immediate early, HSV thymidine kinase, early and late SV40, LTRs from retrovirus, and mouse metallothionein-I. Selection of the appropriate vector and promoter is well within the level of ordinary skill in the art. The expression vector also contains a ribosome binding site for translation initiation and a transcription terminator. The vector may also include appropriate sequences for amplifying expression. Promoter regions can be selected from any desired gene using CAT (chloramphenicol transferase) vectors or other vectors with selectable markers. In addition, the expression vectors can contain one or more selectable marker genes to provide a phenotypic trait for selection of transformed host cells such as dihydrofolate reductase or neomycin resistance for eukaryotic cell culture, or tetracycline, ampicillin, or kanamycin resistance for E. coli, or zeocin or auxotrophic markers for P. pastoris or Gentamycin, Amphotericin B, Penicillin Streptomycin for Sf9, Sf21 and High Five insect cells (Invitrogen)

In one aspect, expression cassettes of the invention comprise a sequence of the invention and a nucleotide sequence which is capable of affecting expression of a structural gene (i.e., a protein coding sequence) in a host compatible with such sequences. Expression cassettes include at least a promoter operably linked with the polypeptide coding sequence; and, optionally, with other sequences, e.g. transcription termination signals. Additional factors necessary or helpful in effecting expression may also be used, e.g., enhancers. In one aspect, “operably linked” as used herein refers to linkage of a promoter upstream from a DNA sequence such that the promoter mediates transcription of the DNA sequence. Thus, expression cassettes also include plasmids, expression vectors, recombinant viruses, any form of recombinant naked DNA vector, and the like. In one aspect, a “vector” comprises a nucleic acid that can infect, transfect, transiently or permanently transduce a cell. A vector can be a naked nucleic acid, or a nucleic acid complexed with protein or lipid.

A DNA sequence may be inserted into a vector by a variety of procedures. In general, the DNA sequence is ligated to the desired position in the vector following digestion of the insert and the vector with appropriate restriction endonucleases. Alternatively, blunt ends in both the insert and the vector may be ligated. A variety of cloning techniques are known in the art.

The invention relates also to transformed cells comprising a nucleic acid sequence according to the present invention, e.g., a sequence encoding a variant of a polysaccharide lyase according to the invention, or comprising an expression cassette, vector, cloning vehicle, expression vector, or cloning vector of the invention. The host cell may be any of the host cells familiar to those skilled in the art, including prokaryotic cells, eukaryotic cells, such as bacterial cells, fungal cells, yeast cells, mammalian cells, insect cells, or plant cells. Exemplary bacterial cells include any species within the genera Escherichia, Bacillus, Streptomyces, Salmonella, Pseudomonas, Lactococcus, and Staphylococcus, including, e.g., Escherichia coli, Lactococcus lactis, Bacillus subtilis, Bacillus cereus, Salmonella typhimurium, Pseudomonas fluorescens. Exemplary fungal cells include any species of Aspergillus, including Aspergillus niger. Exemplary yeast cells include any species of Pichia, Hansenula, Saccharomyces, Schizosacchammyces, or Schwanniomyces, including Pichia pastoris, Saccharomyces cerevisiae, or Schizosaccharomyces pombe. Exemplary insect cells include any species of Spodoptera or Drosophila, including Drosophila S2 and Spodoptera S/P. Exemplary insect cells include Drosophila S2 and Spodoptera Sf9 and Sf21, which are traditional cell lines used with baculovirus and originated at the USDA Insect Pathology Laboratory. The cell lines are also suitable for use in the InsectSelect™ System. These two cell lines originated from the IPLBSF-21 cell line, derived from the pupal ovarian tissue of the fall army worm, Spodoptera frugiperda (O'Reilly et al., 1992; Vaughn et al., 1977). In addition The High Five cell line (BTI-TN-5B1-4, Invitrogen) was developed by the Boyce Thompson Institute for Plant Research, Ithaca, N.Y. and

originated from the ovarian cells of the cabbage looper, Trichoplusia ni (Davis et al., 1992; Granados et al., 1994; Wickham et al., 1992; Wickham and Nemerow, 1993). Furthermore, HPg, IZD-MB0503, IPLB-TN-R², IPLBSF-1254, IPLB-LdEIta, CM-1, IPLB-HvT1, IPLB-SF21AE, IPLB-SF21AE-15, IPLB-SF-1254, and BCIRL-PX2-HNV3 insect cell lines may be used.

Exemplary animal cells include CHO, COS or Bowes melanoma or any mouse or human cell line. The selection of an appropriate host is within the abilities of those skilled in the art.

The vector may be introduced into the host cells using any of a variety of techniques, including transformation, transfection, transduction, viral infection, gene guns, or Ti-mediated gene transfer. Particular methods include calcium phosphate transfection, DEAE-Dextran mediated transfection, lipofection, or electroporation.

Where appropriate, the engineered host cells can be cultured in conventional nutrient media modified as appropriate for activating promoters, selecting transformants or amplifying the genes of the invention. Following transformation of a suitable host strain and growth of the host strain to an appropriate cell density, the selected promoter may be induced by appropriate means (e.g., temperature shift or chemical induction) and the cells may be cultured for an additional period to allow them to produce the desired polypeptide or fragment thereof.

The expressed and secreted variants can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. The constructs in host cells can be used in a conventional manner to produce the gene product encoded by the recombinant sequence. Depending upon the host employed in a recombinant production procedure, the polypeptides produced by host cells containing the vector may be glycosylated or may be nonglycosylated. Polypeptides of the invention may or may not also include an initial methionine amino acid residue.

Cell-free translation systems can also be employed to produce a polypeptide of the invention. Cell-free translation systems can use mRNAs transcribed from a nucleic acid construct comprising a promoter operably linked to a nucleic acid encoding the polypeptide or fragment thereof. In some aspects, the nucleic acid construct may be linearized prior to conducting an in vitro transcription reaction. The transcribed mRNA is then incubated with an appropriate cell-free translation extract, such as a rabbit reticulocyte extract, to produce the desired polypeptide or fragment thereof.

Host cells comprising the polynucleotides of interest can be cultured in conventional nutrient media modified as appropriate for, activating promoters, selecting transformants or amplifying genes. The culture conditions, such as temperature, pH and the like, are those previously used with the host cell selected for expression, and will be apparent to the ordinarily skilled artisan. The clones which are identified as having the specified enzyme activity may then be sequenced to identify the polynucleotide sequence encoding an enzyme having the enhanced activity.

Yet another aspect of the present invention relates to a method for the recombinant production of a polysaccharide lyase variant having a beta helix structure according to the present invention comprising the steps of:
a) cultivating a host cell comprising a nucleic acid molecule or a vector according to the present invention in a culture medium,
b) isolating the polysaccharide lyase variant from the cell culture.

The isolation of the pectate lyase variants from the cell culture can be peformed either from the cell biomass or cell pellet (in case of intracellular cytoplasmic or periplasmic expression) or from the supernatant of the cell culture supernatant or the cell culture medium (in case of extracellular secretion)

According to a preferred embodiment of the present invention the pectate lyase variant is secreted into the culture medium. In another preferred embodiment, the secretion signal sequence is removed from the polysaccharide lyase variant or polypeptide by incubating the lyase variant or polypeptide of step b) to a protease capable to cleave the secretion signal sequence from the lyase variant of a polypeptide. Respective methods are well known to the person skilled in the art.

Another aspect of the present invention relates to a method for identifying a variant according to the present invention, wherein the N- and/or C-terminal truncation of the polysaccharide lyase is identified by
(i) determining the two and three dimensional structure of the polysaccharide lyase,
(ii) identifying the beta helix structure,
(iii) determining the first and last beta sheet being part of the beta helix structure,
(iv) truncating the region extending from the N-terminal end of the polysaccharide lyase to approximately 1 to 50 amino acid residues before the N-terminus of the first beta sheet being part of the beta helix structure of said variant and the region extending from the C-terminal end of the polysaccharide lyase to approximately 1 to 50 amino acid residues after the C-terminus of the last beta sheet part of the beta helix structure, under the provisio that the first and last beta sheet of the beta helix structure remains intact.

The single methods identified above are well known in the art.

The present invention relates also to the following embodiments:

1. A variant, preferably a hypoallergenic variant, of a mature polysaccharide lyase having a beta helix structure, wherein said variant comprises an N- and/or C-terminal truncation and consists essentially or of at least 90% of the beta helix structure of the mature polysaccharide lyase.
2. Variant according to embodiment 1, wherein the N- and/or C-terminal truncation comprises at least 10 amino acid residues.
3. Variant according to embodiment 1 or 2, wherein said N-terminal truncation comprises the region extending from the N-terminal end of the polysaccharide lyase to approximately 1 to 50 amino acid residues before the N-terminus of the first beta sheet being part of the beta helix structure and said C-terminal truncation comprises the region extending from the C-terminal end of the polysaccharide lyase to approximately 1 to 50 amino acid residues after the C-terminus of the last beta sheet part of the beta helix structure, under the provisio that the first and last beta sheet of the beta helix structure remains intact.
4. Variant according to any one of embodiments 1 to 3, wherein the polysaccharide lyase is a member of a family of proteins selected from the group consisting of pectate lyase, hyaluronate lyase, poly(β-D-mannuronate) lyase, chondroitin AC lyase, oligogalacturonide lyase, heparin lyase, heparin-sulfate lyase, pectate disaccharide-lyase, pectin lyase, poly(α-L-guluronate) lyase, xanthan lyase, exo-(1-->4)-α-D-glucan lyase, glucuronan lyase, anhydrosialidase, levan fructotransferase, inulin fructotransferase, chondroitin B lyase, chondroitin-sulfate-ABC endolyase, chondroitin-sulfate-ABC exolyase and pectate trisaccharide-lyase.
5. Variant according to embodiment 4, wherein the polysaccharide lyase is a pectate lyase, preferably an allergenic pectate lyase.
6. Variant according to embodiment 5, wherein the allergen is selected from the group consisting of Amb a 1, Art v 6, Cha o 1, Cup a 1, Cry j 1 and Jun a 1.
7. Variant according to embodiment 5 or 6, wherein the variant is hypoallergenic.
8. Variant according to embodiment 7, wherein the variant derived from Amb a 1 consists essentially of amino acid sequence SEQ ID No: 1, the variant derived from Cry j 1 consists essentially of amino acid sequence SEQ ID No: 2, the variant derived from Cup a 1 consists essentially of amino acid sequence SEQ ID No: 3, the variant derived from Jun a 1 consists essentially of amino acid sequence SEQ ID No: 4, the variant derived from Cha o 1 consists essentially of amino acid sequence SEQ ID No: 5 and the variant derived from Art v 6 consists essentially of amino acid sequence SEQ ID No: 6.
9. Polypeptide comprising a secretion signal sequence fused directly or via a linker to a variant according to any one of embodiments 1 to 8.
10. Polypeptide according to embodiment 9, wherein the secretion signal sequence is of bacterial, fungal, insect or mammalian origin.
11. Polypeptide according to embodiment 9 or 10, wherein the secretion signal sequence is selected from the group consisting of a PelB, a PelC, a OmpA, a StII, an EX, a PhoA, a OmpF, a PhoE, a MalE, an OmpC, a LPP, a LamB, an OmpT, a LTB, ecdysteroid UDPglucosyltransferase (egt) and the envelope glycoprotein gp67, a phosphatase (phol) and an invertase (Sue) secretion signal sequence and an α-mating factor prepro-sequence.
12. Polypeptide according to any one of embodiments 9 to 11, wherein the linker comprises a protease cleavage site.
13. Polypeptide according to embodiment 12, wherein the protease cleavage site is capable of being cleaved by thrombin, chymotrypsin, trypsin, pepsin, subtilisin, enterokinase, factor Xa, TEV protease and PreScission protease, whereby TEV protease is particularly preferred.
14. Nucleic acid molecule encoding a variant of a polysaccharide lyase according to any one of embodiments 1 to 8 or a polypeptide according to any one of embodiments 9 to 13.
15. Nucleic acid molecule according to embodiment 14, wherein said nucleic acid molecule comprises further a nucleotide sequence encoding for a leader sequence.
16. Nucleic acid molecule according to embodiment 15, wherein the leader sequence is selected from the group consisting of PelB, PelC and α-mating factor prepro-sequence.
17. Nucleic acid molecule according to any one of embodiments 14 to 16, wherein the nucleotide sequence of said nucleic acid molecule is codon optimised and/or codon harmonised.
18. Nucleic acid molecule according to any one of embodiments 14 to 17, wherein the nucleic acid molecule encoding the variant of Amb a 1 has the nucleotide sequence SEQ ID No: 7 or SEQ ID No: 8, the nucleic acid molecule encoding the variant of Cry j 1 has the nucleotide sequence SEQ ID No: 9 or 10, the nucleic acid molecule encoding the variant of Cup a 1 has the nucleotide sequence SEQ ID No: 11 or 12, the nucleic acid molecule encoding the variant of Art v 6 has the nucleotide sequence SEQ ID No: 13 or 14, the nucleic acid molecule encoding the variant of Jun a 1 has the nucleotide sequence SEQ ID No: 15 or 16 and the nucleic acid molecule encoding Cha o 1 has the nucleotide sequence SEQ ID No: 17 or 18.
19. Vector comprising a nucleic acid molecule according to any one of embodiments 14 to 18.
20. Method for the recombinant production of a polysaccharide lyase variant having a beta helix structure according to any one of embodiments 1 to 8 or a polypeptide according to any one of embodiments 9 to 13 comprising the steps of:
a) cultivating a host cell comprising a nucleic acid molecule according to any one of embodiments 14 to 18 or a vector according to embodiment 19 in a culture medium,
b) isolating the polysaccharide lyase variant or polypeptide from the cell culture.
21. Method according to embodiment 20, wherein the polysaccharide lyase variant or polypeptide is isolated from the cell biomass.
22. Method according to embodiment 20, wherein the polysaccharide lyase variant or polypeptide is isolated from the cell culture supernatant or the cell culture medium.
23. Method according to embodiment 20, wherein the polysaccharide lyase variant or polypeptide is secreted by the cells, and the secretion signal sequence is removed from the polysaccharide lyase variant or polypeptide by incubating the lyase variant or polypeptide of step b) to a protease capable to cleave the secretion signal sequence from the lyase variant of a polypeptide.
24. Method according to any one of embodiments 20 to 23, wherein the host cell is Escherichia coli.
25. Method according to any one of embodiments 20 to 23, wherein the host cell is Pichia pastoris.
26. Vaccine formulation for the treatment of an allergy comprising at least one of the variants according to embodiments 5-7, further comprising at least one adjuvant, pharmaceutical acceptable excipient and/or preservative.

The present invention is further illustrated in the following figures and examples, however, without being restricted thereto.

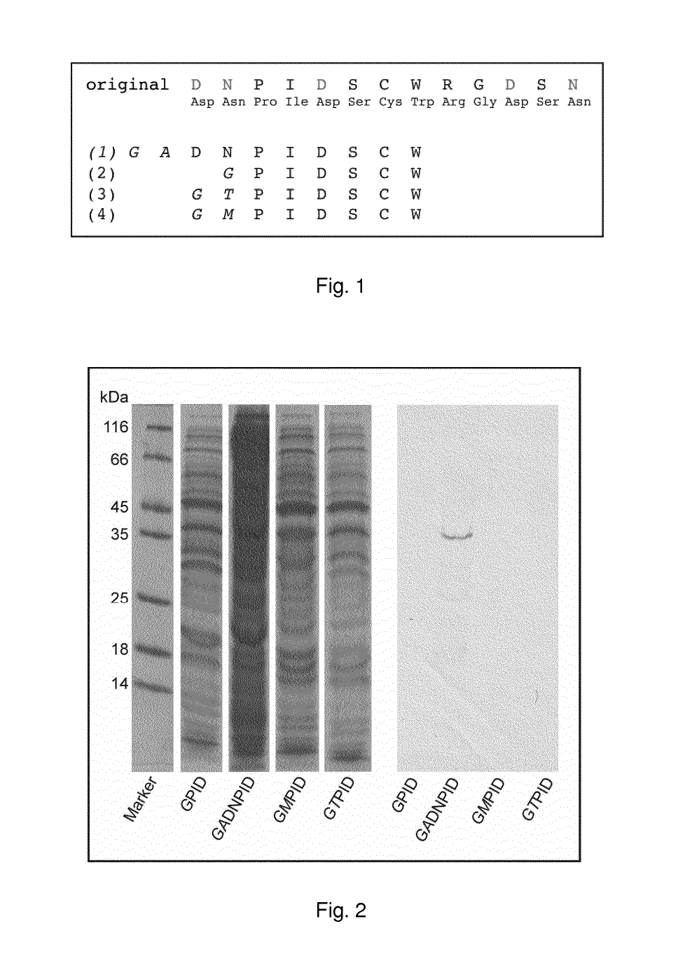

FIG. 1 shows Cry j 1 with modified amino termini. (1) with additional Gly-Ala. (2) deletion of Asp-Asn, instead Gly. (3) exchange Asp-Asn to Gly-Thr. (4) exchange Asp-Asn to Gly-Met.

FIG. 2 shows a soluble bacterial protein fraction after expression in E. coli. Coomassie-stained SDS-PAGE and Wetsern Blot using a murine monoclonal anti Cry j 1 antibody.

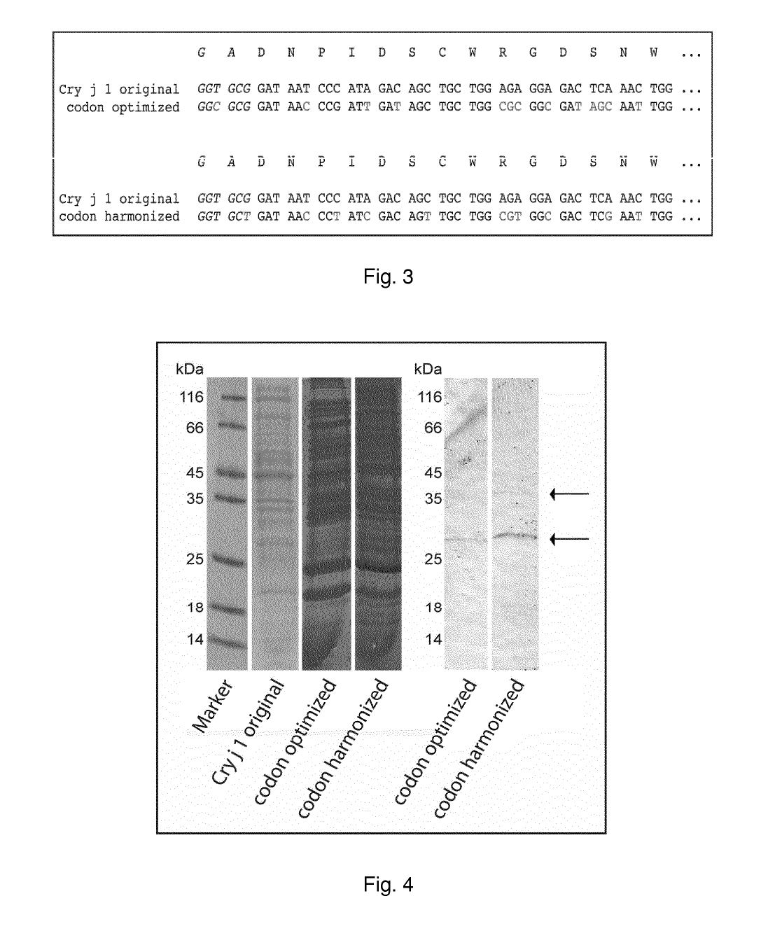

FIG. 3 shows the differences in N-terminal codon usage after codon optimization and codon harmonization of Cry j 1, including additional Gly-Ala at the amino terminus.

FIG. 4 shows SDS-PAGE and Western Blot using murine anti Cry j 1 monoclonal detection antibody of Cry j 1 constructs expressed in E. coli BL21 (DE3) (Novagen) for 24 h at 37° C. using auto-induction media.

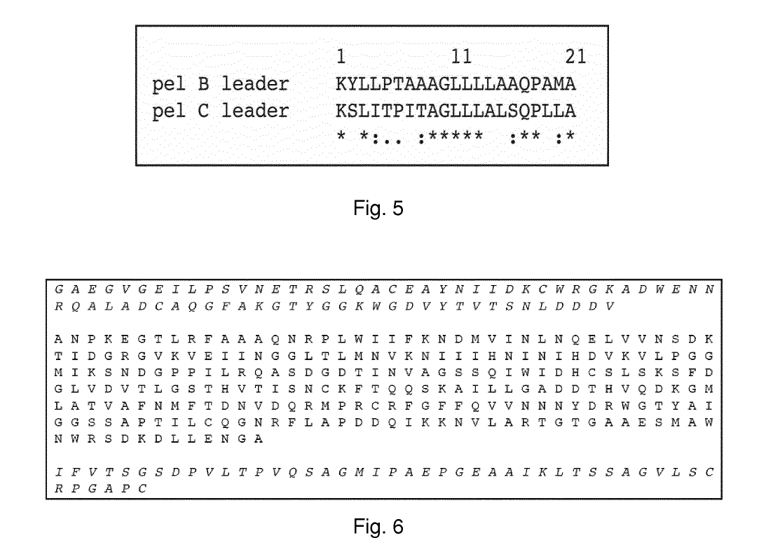

FIG. 5 shows the difference between pET26-b vector pelB leader sequence and Erwinia chrysanthemi pelC leader sequence.

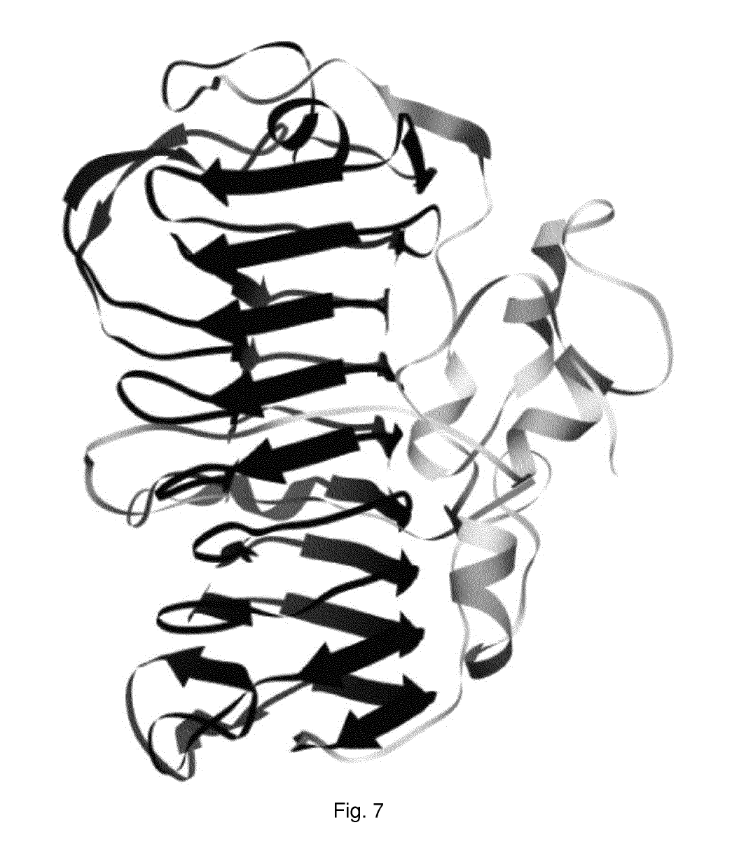

FIG. 6 shows the sequence of Amb a 1.3 R2. Sequence areas depicted in italic are lacking, resulting the truncated Amb a 1.3 R2.

FIG. 7 shows Amb a 1.3 R2 truncated model in black. Crystal structure of natural Jun a 1 (PDB 1pxz) was used as template structure.

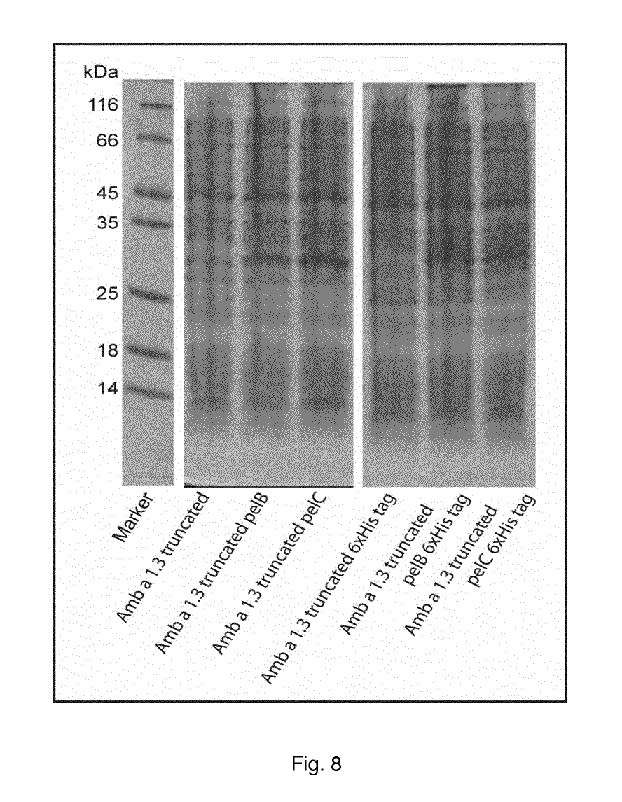

FIG. 8 shows whole cell protein extract of Amb a 1.3 R2 truncate constructs expressed in E. coli BL21 (DE3) star in LBsupp media at 16° C. for 20 h.

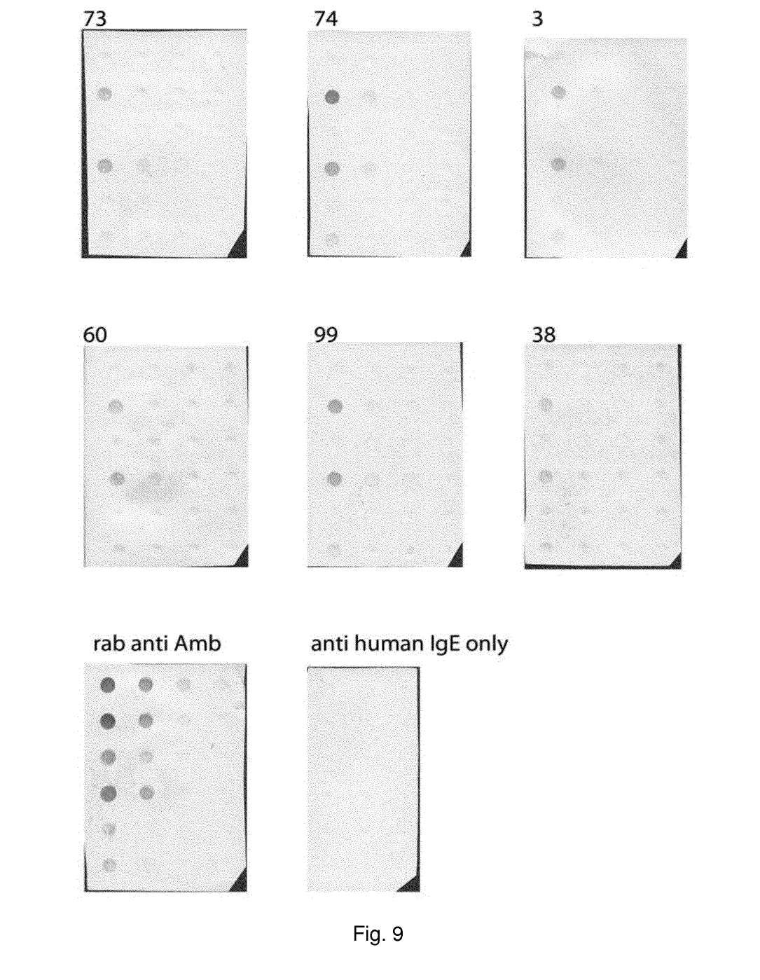

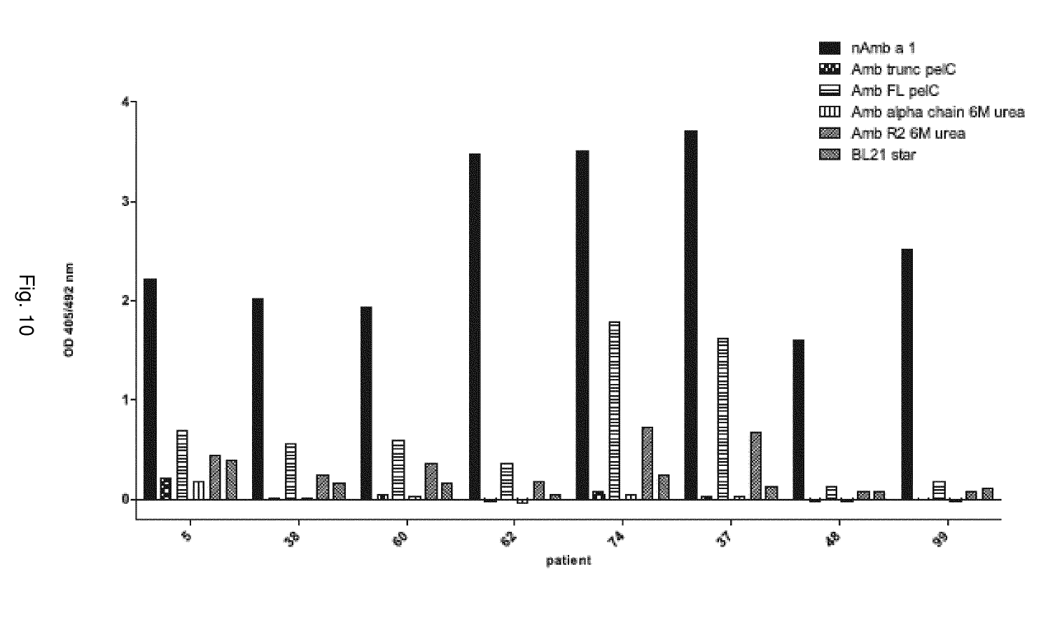

FIG. 9 shows dot blot analyses of Amb a 1 and derivatives thereof expressed in E. coli. E. coli transfected with the empty expression plasmid as well as purified natural Amb a 1 were used as reference.

FIG. 10 shows ELISA analyses of Amb a 1 and derivatives thereof expressed in E. coli. E. coli transfected with the empty expression plasmid as well as purified natural Amb a 1 were used as reference.

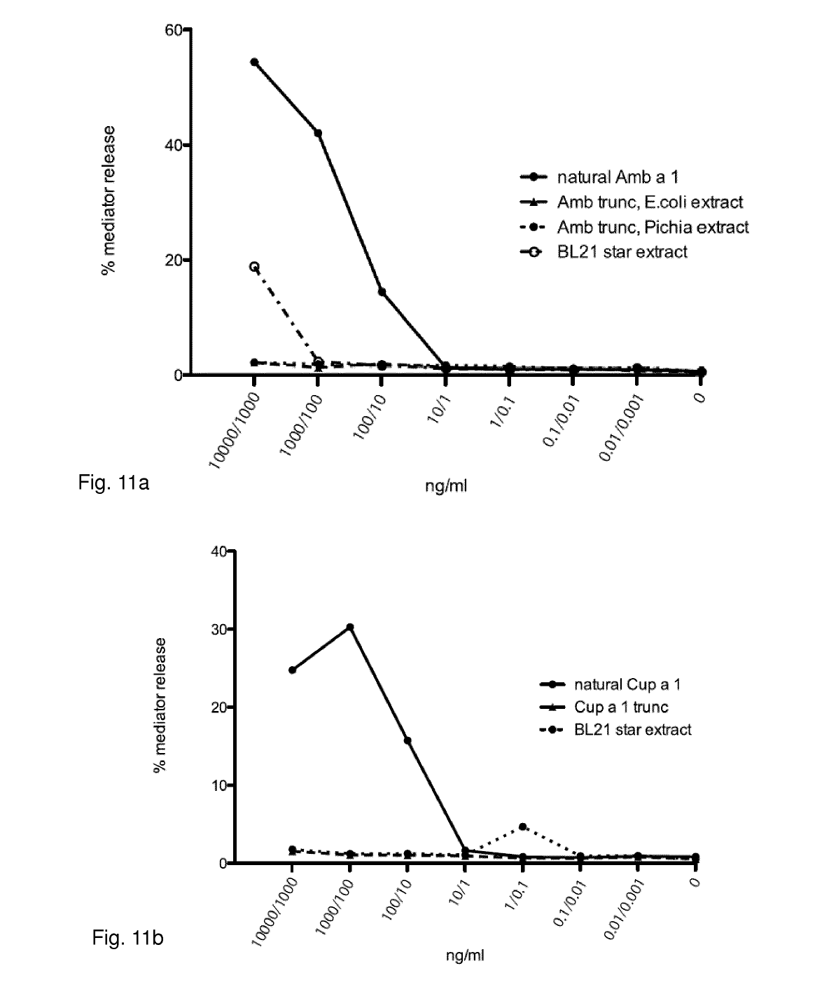

FIG. 11 shows mediator release analyses of Amb a 1 and derivatives thereof expressed in E. coli and in P. pastoris (a) and Cup a 1 (b). E. coli transfected with the empty expression plasmid as well as purified natural Amb a 1 were used as reference.

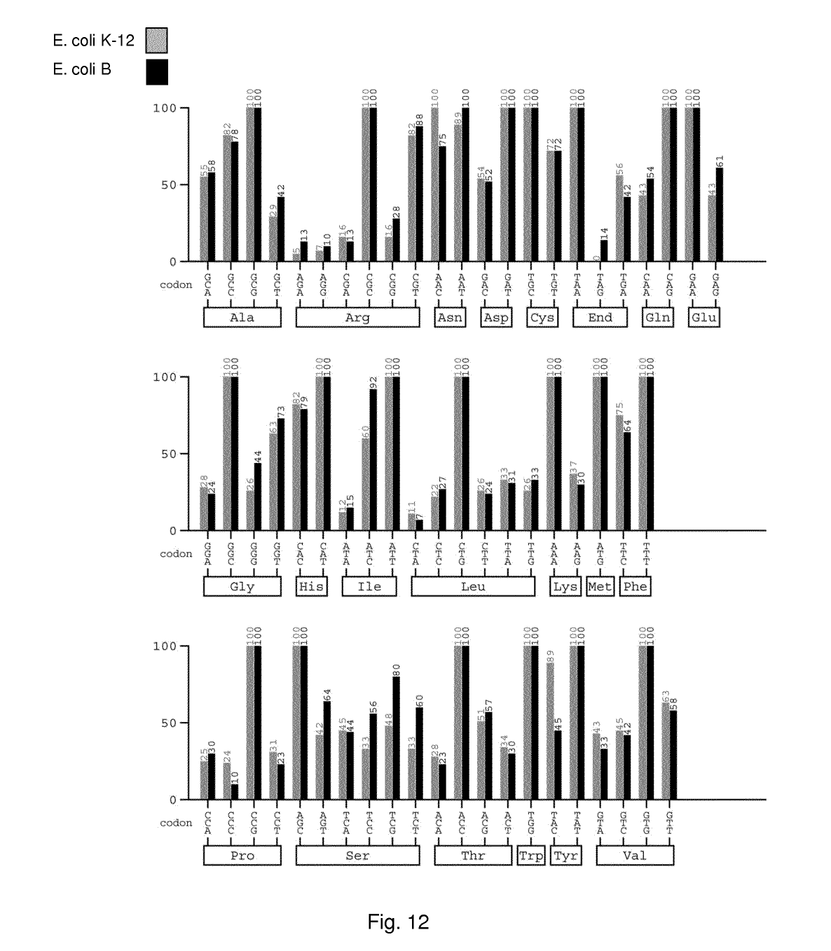

FIG. 12 shows the comparison of codon usages between E. coli K-12 and E. coli B.

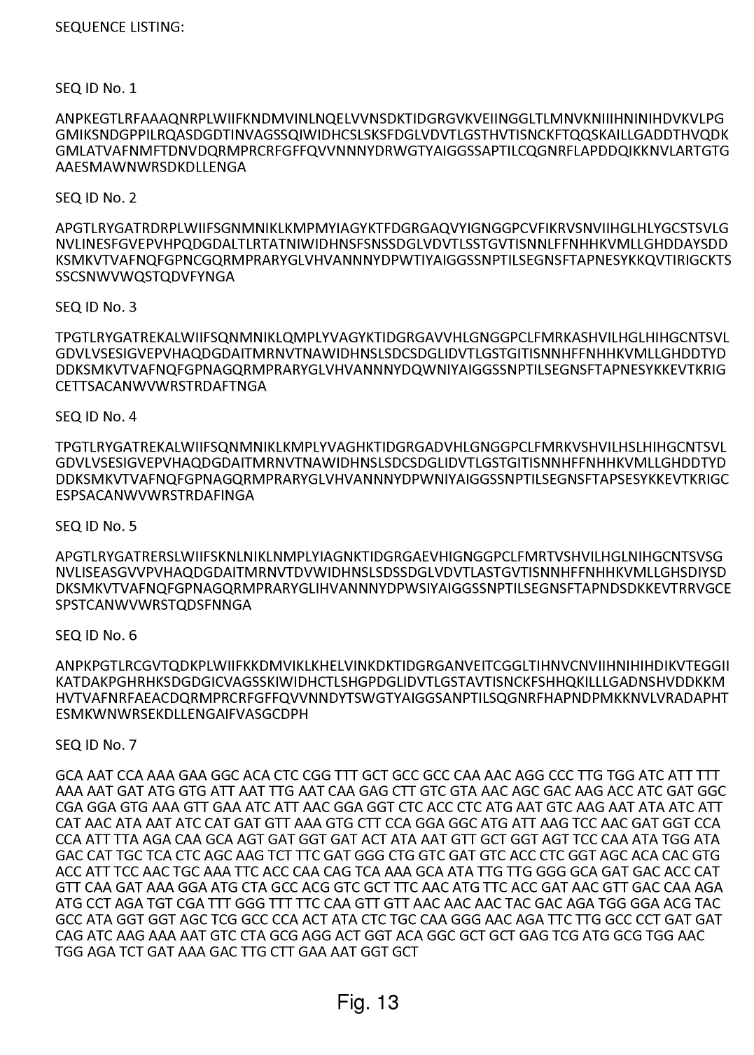

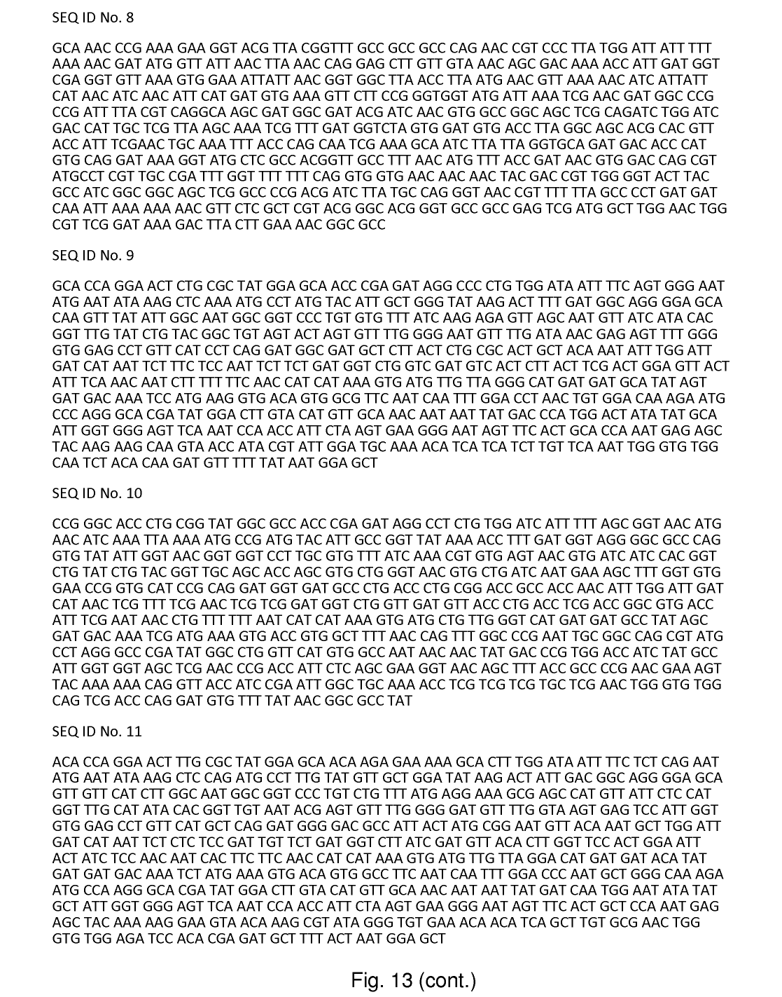

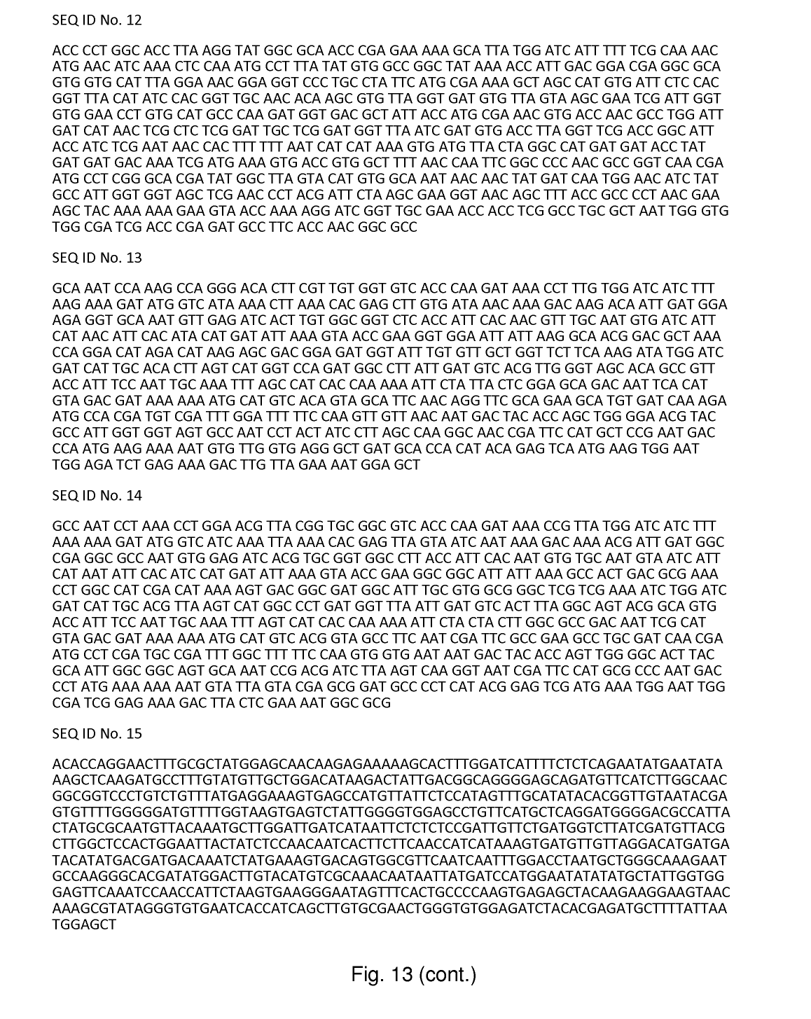

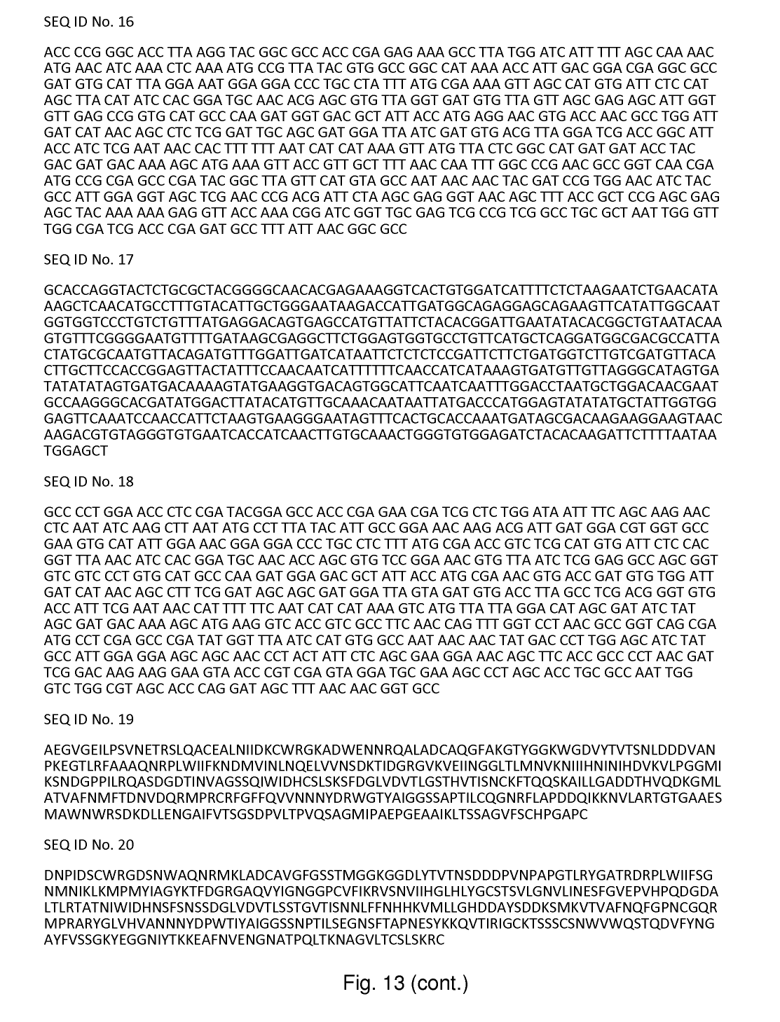

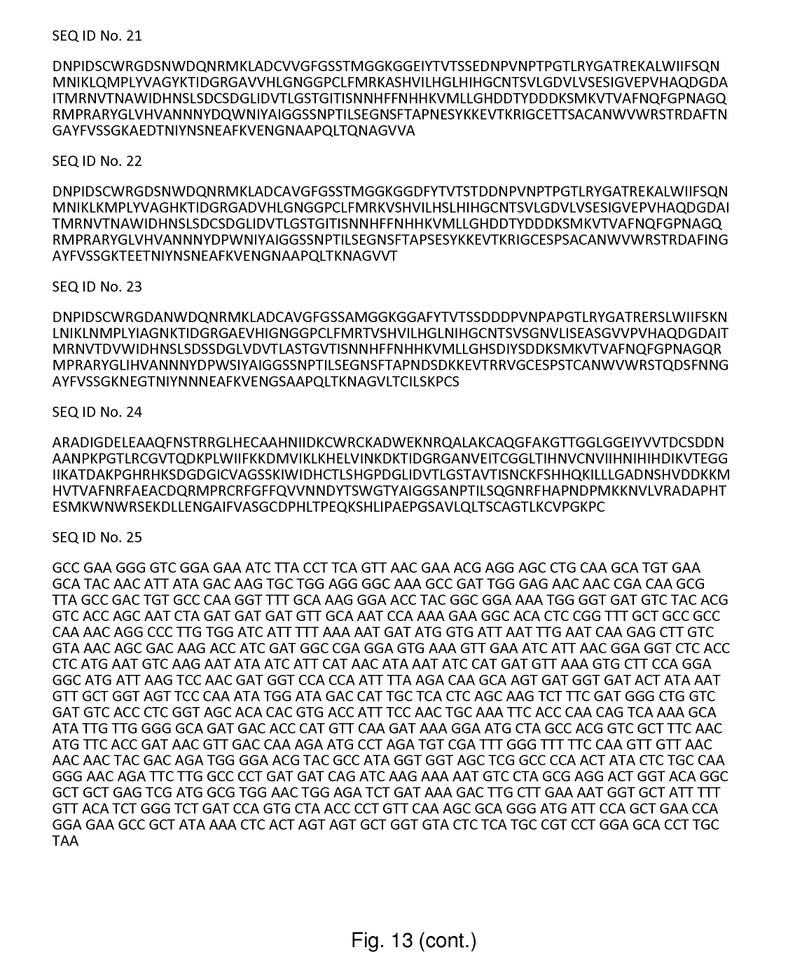

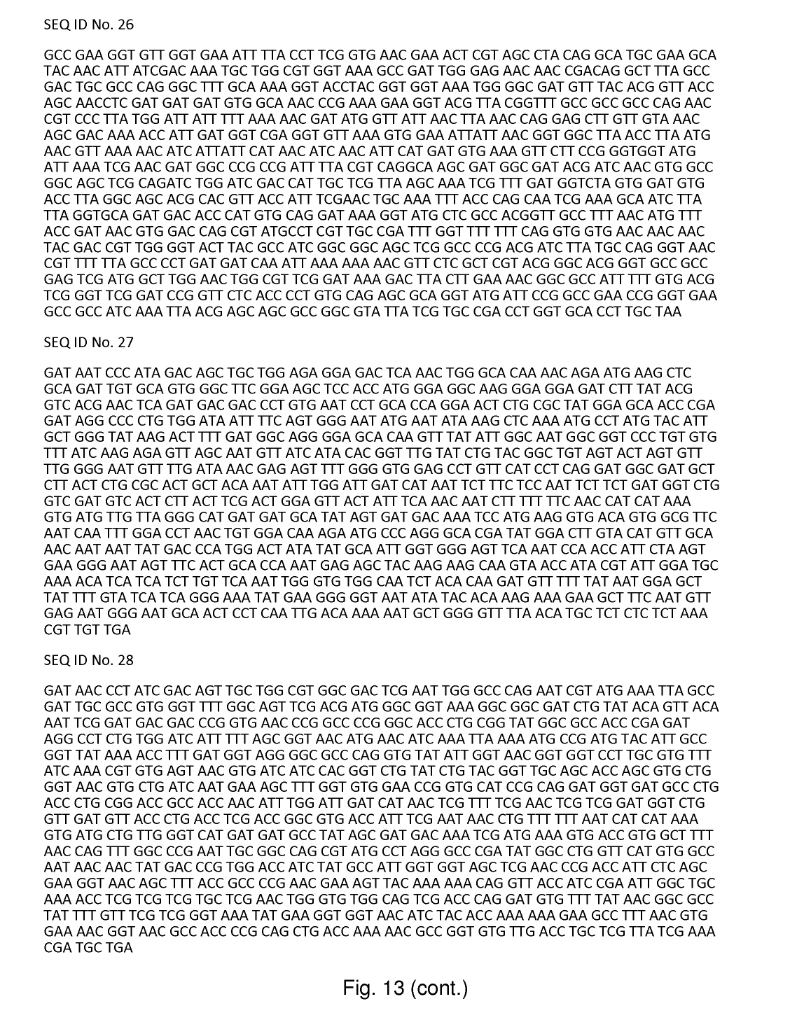

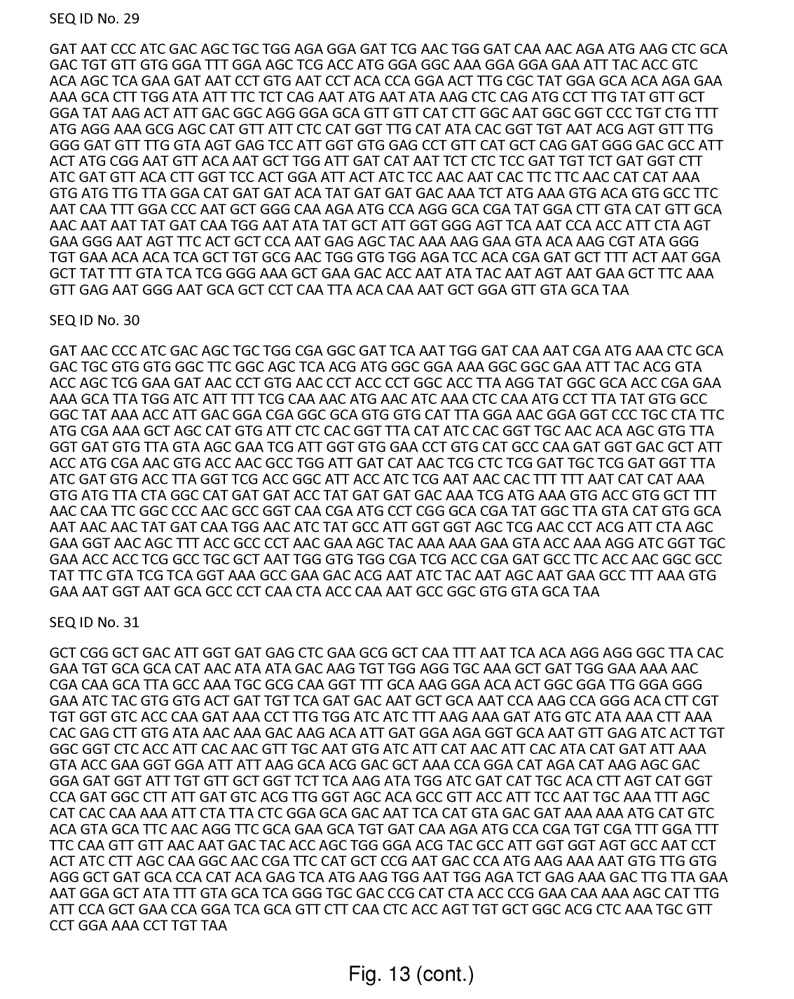

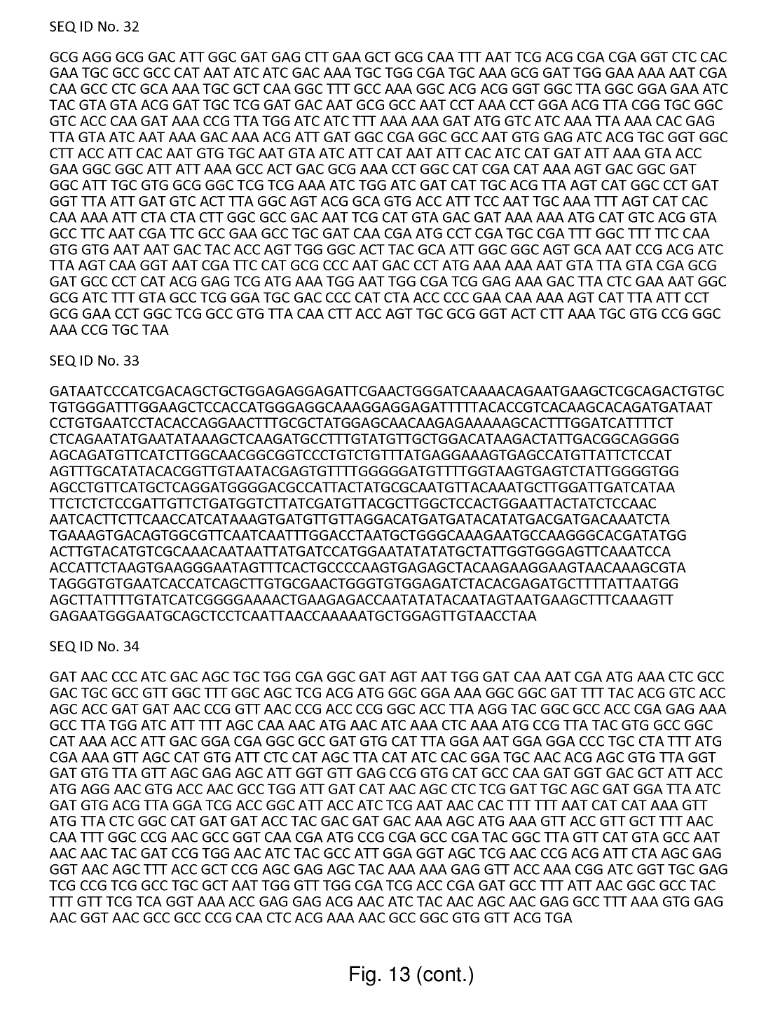

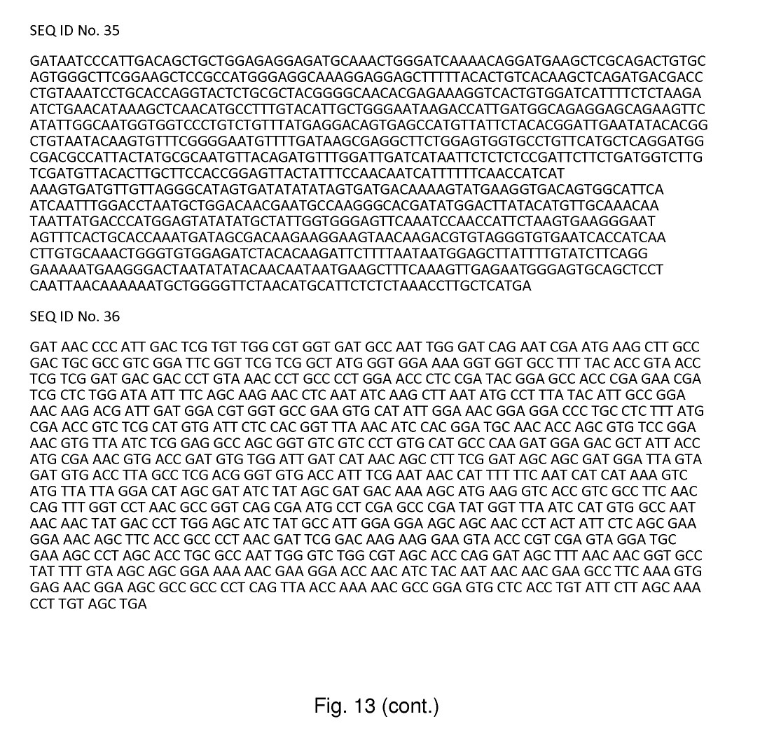

FIG. 13 shows the sequences associated to their respective sequence identifiers (SEQ ID Nos.).

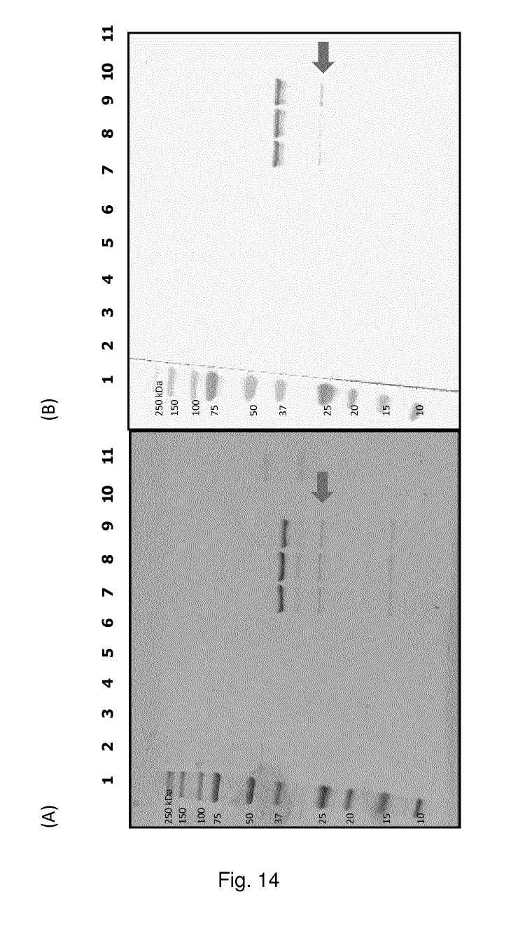

FIG. 14 shows an SDS-PAGE (A) and Western Blot (B) from a cell supernatant of Amb a 1.3 truncated (according to SEQ ID No: 1) secreted by several clones of Pichia pastoris. Data are from shake flask cultivation. The arrow indicates the intended Amb a 1.3. truncated molecule (expected molecular weight: 27.7 kDa) n methanol-free (lane 2-5) or methanol-induced strains (lane 6-11) by SDS-Page (A) and Westernblot (B).

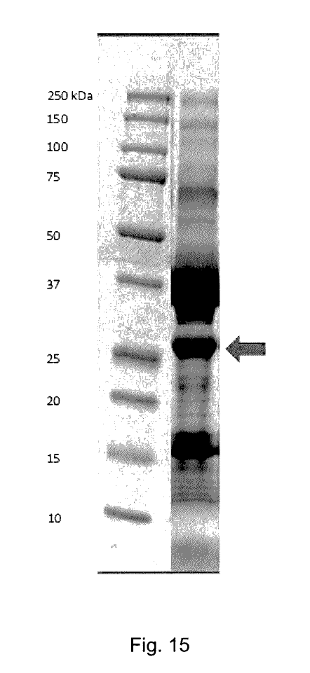

FIG. 15 shows an SDS-PAGE from a cell supernatant of Amb a 1.3 truncated (according to SEQ ID No: 1) of Pichia pastoris cultivated in a bioreactor (fermenter). The arrow indicates the intended Amb a 1.3. truncated molecule (expected molecular weight: 27.7 kDa).

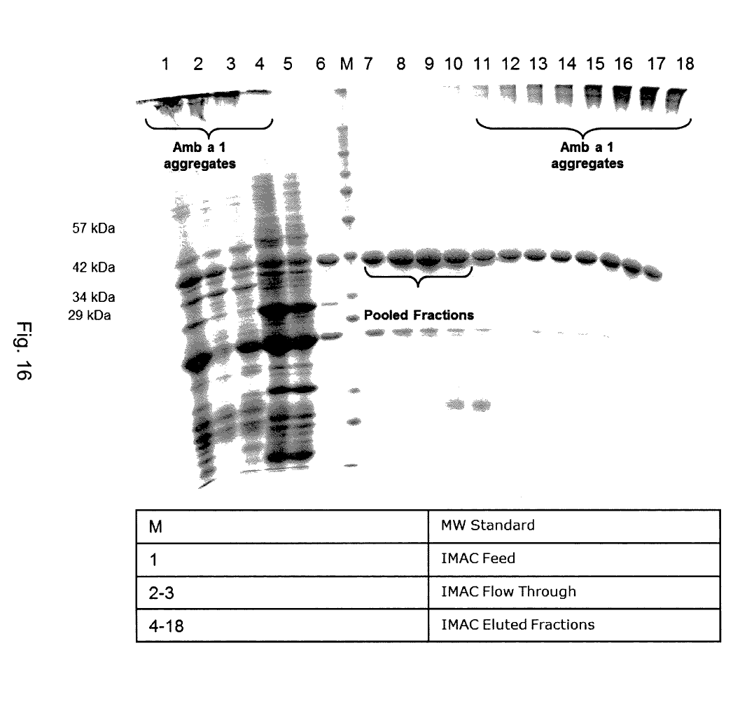

FIG. 16: SDS-PAGE from IMAC eluate fractions containing recombinant wild-type Amb a 1 showing high molecular weight aggregates at the top of the gel slots in columns 1-4 and 11-18.

### EXAMPLES

### Example 1

### Expression and Characterization of Pectate Lyase Variants According to the Invention

### Material and Methods

### Patients and Sera.

Ambrosia artemisiifolia, Cryptomeria japonica, Juniperus ashei, or Cupressus arizonica pollen-allergic patients were selected based on case history, positive in vivo skin prick test or in vitro IgE detection (CAP system, Phadia AB).

Codon optimization of Amb a 1 and Cry j 1.

The codon usage for a given organism is available at the codon usage database http://www.kazusa.or.jp/codon/. Codon optimization of Amb a 1 and Cry j 1 were performed according to published methods (Gustafsson C. et al., Trends Biotechnol. 2004 July; 22(7):346-53). Most of the commonly used commercially available E. coli strains are derivatives of E. coli B (e.g. BL21 and derivatives thereof, Tuner™, Origami B, Rosetta-Gami B, all available at EMD Biosciences) or E. coli K-12 (e.g. Origami 2, Rosetta 2, Rosetta-Gami 2, all available at EMD Biosciences). For protein expression the strains BL21 Star (DE3) (Invitrogen), an E. coli B derivative, and Origami 2 (EMD Biosciences), an E. coli K-12 derivative, were selected. E. coli B and K-12 are closely related (Lukjancenko O. et al., Microb Ecol. 2010 November; 60(4):708-20) and the codon usage of both strains is comparable. A graphical comparison of both codon usage tables was performed by a graphical codon usage analyzer (http://gcua.schoedl.de/compare_v2.html) (FIG. 12). For the codon optimization the codon usage of E. coli K-12 was used and the genes of interest (e.g. Amb a 1, and Cry j 1) were codon optimized accordingly.

Codon harmonization of Amb a 1 and Cry j 1.

The codon usage for a given organism is available at the codon usage database http://www.kazusa.or.jp/codon/. Besides codon optimization, codon harmonization has been demonstrated to be an effective tool to increase protein expression and solubility (Angov E. et al., PLoS One. 2008 May 14; 3(5):e2189.; Marin M. Biotechnol J. 2008 August; 3(8):1047-57.). In brief, rare codons slow down the translation of the mRNA allowing the newly synthetized polypeptide chain to adopt the correct conformation, thus directly affecting the folding, the structure and the biological activity of proteins. Therefore the codon usage of pectate lyase genes was determined for the respective source organism. The codon usage of the host organism was used to transfer the codon usage for the gene of interest from the source organism to the host organism. Therefore, E. coli codons encoding the respective amino acid at a given position and showing the codon usage, which was closest to the original codon of the source organism for the respective amino acid, were selected. The DNA code was then changed accordingly and the resulting gene with new codon composition was generated synthetically. For protein expression the strains BL21 Star (DE3) (Invitrogen), an E. coli B derivative, and Origami 2 (EMD Biosciences), an E. coli K-12 derivative, were selected. For the codon harmonization the codon usage of E. coli K-12 was used and the genes of interest (e.g. Amb a 1, and Cry j 1) were codon harmonized accordingly (see FIG. 12 a to e). Cloning and expression of pectate lyases and derivatives thereof.

### Expression and Purification of Pectate Lyase Variants in E. Coli

Pectate lyase genes were PCR amplified for the original cloning vectors and subsequently cloned into pET vectors (EMD Biosciences) using the respective restriction sites. Cells were grown in LB medium to an OD600 of 0.8 at 37° C. and 200 rpm on a shaker, and protein expression in was induced by the addition of 0.5 M isopropyl-β-D-thiogalactopyranosid. Expression was performed at various growth conditions (e.g. 37° C. for 6 h, 16° C. for 20 h) using different expression constructs as well as E. coli host strains. Thereafter, cells were harvested by low-speed centrifugation, resuspended in 25 mM TrisCl pH 9.5, 0.5 M Urea and subjected to three repeated freeze/thaw cycles at 37° C. and liquid nitrogen, respectively. Thereafter, the suspension was equilibrated for 1 h at room temperature on a low-speed shaker, homogenized with an Ultra-turrax for 1 min at 14.000 rpm, and centrifuged at 20.000 g for 20 min at 4° C. Soluble and insoluble protein fractions were collected for further analysis.

Expression and Secretion of Polysaccharide Lyase Variant (Amb a 1.3 Truncated) with Pichia pastoris

The synthetically optimized DNA sequence (codon optimizsation) coding for polysaccaride lyase (pectate lyase) variant Amb a 1.3 truncated was cloned into a standard expression vector suitable for Pichia pastoris(pPICZα, Invitrogen; AOX1 promoter, secretion leader signal α-mating factor). After transformation of the linearized expression vector into competent Pichia pastoris host cells and the genomic integration of the expression vector, a number of clones were screened (microwell plates) and finally selected with respect to their product formation and secretion performance (expression level of Amb a 1.3. truncated).

Four single colonies of transformed cells were culitvated in shake flasks, and Amb a 1.3 truncated expression was tested in absence and presence of methanol. As shown in FIG. 14, secreted recombinant Amb a 1.3 truncated was found in the culture supernatant of methanol-induced cultures, detected by SDS-PAGE (A) and by Western Blot (B), see arrow (expected molecular weight of Amb a 1.3 truncated: 27.7 kDa).

### ELISA Experiments.

For ELISA experiments, Maxisorp plates (Nalge Nunc, Rochester, N.Y., USA) were coated with protein antigen (200 ng/well in 50 μl PBS) overnight at 4° C. Plates were blocked with Tris-buffered-saline (TBS), pH 7.4, 0.05% (v/v) Tween, 1% (w/v) bovine serum albumin (BSA) and incubated with patients' sera diluted 1:10 overnight at 4° C. Bound IgE was detected with alkaline phosphatase-conjugated monoclonal anti-human IgE antibodies (BD Biosciences), after incubation for 1 h at 37° C. and 1 h at 4° C. 10 mM 4-nitrophenyl phosphate (Sigma-Aldrich) was used as substrate and OD was measured at 405/492 nm. All measurements were performed as duplicates; results are presented as mean OD values.

### Activation of Basophils.

The allergenic potential of Amb a 1 and derivatives thereof expressed in E. coli and P. pastoris, respectively (FIG. 11a), as well as Cup a 1 and derivatives thereof (FIG. 11b) was measured by rat basophil degranulation assay (RBL) as described (Vogel L. et al., Allergy. 2005 August; 60(8):1021-8.). A serum pool containing sera from ragweed pollen allergic human individuals was added in a dilution of 1:15, antigen solutions were titrated and antigen-dependent beta-hexosaminidase release into the supernatant was measured by enzymatic cleavage of the fluorogenic substrate 4-methyl umbelliferyl-N-acetyl-β-glucosaminide and expressed as % of total enzyme content of Triton X100-treated cells.

### Results

### Second Amino Acid Position

The half-life of a cytosolic protein is determined to a large extent by its amino-terminal residue. A highly destabilizing residue (e.g. arginine, asparagine, aspartic acid, glutamine, glutamic acid, and leucine) favors rapid ubiquitinylation and therefore, destruction of the expressed protein, whereas stabilizing residues do not.

Many plant pectate lyases show a tendency to express such destabilizing amino acid residues at the mature N-terminus. To avoid destruction and to increase the expression level, 4 constructs with altered N-terminal amino acid sequence were designed (see FIG. 1).

The 4 constructs were used for protein expression in E. coli grown after induction with IPTG at OD600 nm 0.7 for 7 hours at 37° C. in LB supplement media. Cell breakage was performed using liquid nitrogen and resulting soluble protein fractions were analyzed by SDS-PAGE and Western Blot (see FIG. 2).

The Cry j 1 construct with additional Gly-Ala at the N-terminus shows higher total protein expression in E. coli, when compared with the other constructs. Furthermore, when using the Cry j 1 construct possessing Alanine at the second amino acid position, expression of recombinant Cry j 1 was clearly detectable via Western Blot.

This observation is in accordance with E. coli protein expression studies, showing that high expressed proteins have an increased frequency of alanine in the second amino acid position.

### Codon Harmonization

The process of codon optimization for protein production is a widely used technique in molecular biology. However, during heterologous protein expression, low expression levels or formation of insoluble aggregates may be attributable to differences in synonymous codon usage between expression and natural host. In contrast, codon harmonization uses synonymous codons from E. coli that match as closely as possible the codon usage frequency used in the native gene. Several studies could demonstrate that the approach of codon harmonization significantly improved protein yield and protein solubility (Angov E, et al. PloSOne 3 (2008):e2189).

Therefore, a codon optimized and a codon harmonized version of the Cry j 1-gene were designed and compared in protein expression levels.

Escherichia coli K-12 codon usage data were used, as Origami and BL21 (DE3) strains used for protein expression are derivatives of strain K-12.

For protein expression, original Cry j 1 as well as Cry j 1 codon optimized and Cry j 1 harmonized were compared (see FIG. 4).

Expression of original, codon optimized, and codon harmonized Cry j 1 was performed using LB media as well as auto-induction media. Improved expression results could be monitored when using auto-induction media and the Cry j 1 codon harmonized construct (see FIG. 4). However, very little amount of the protein expressed seems to be full length Cry j 1; most of the protein is degraded (around 27 kDa in the immunoblot). This seems to be a possible “alpha chain”, as known for the ragweed major allergen Amb a 1 (Wopfner N, et al. Molecular Immunology 46 (2009):2090-2097).

### Leader Sequence

Optimization of the second amino acid position and codon harmonization were shown to afford soluble expression of recombinant Cry j 1 constructs. To increase the protein expression level, an expression vector including a leader sequence for potential periplasmic localization of the recombinbantly produced protein. The vector used was pET26b (Novagen) combining the formerly used pET 28b vector and a pelB leader sequence (pelB from pectate lyase B).

However, additional investigations regarding the family tree of all known pectate lyases revealed allergenic plant pectate lyases like Cry j 1, Amb a 1, and Cup a 1 to be more related with pectate lyases C than pectate lyases B (pectate lyases included are from CAZY—Carbohydrate-active enzymes database, for pectate/pectin lyases n=178; see FIG. 5). Therefore, the pelC leader sequence from Erwinia crysanthemi was used instead of the pelB leader sequence. Furthermore, to investigate if this approach is also applicable for other pectate lyases, Amb a 1 was used as template.

### Truncation

One objective was to generate a pectate layse hypoallergen for use in immunotherapy. However, the so far described approaches afford soluble expression of full length pectate lyases, i.e. Amb a 1, Cry j 1, and Cup a 1.

The family of pectate lyases, independent from which organism, feature one crucial structural similarity, the beta helix. A folding motif composed of three parallel beta strands forming a large right-handed helix, shown to be a very stable structural motif (Yoder M D, et al. The FASEB Journal 9 (1995):335-342). The various known pectate lyases differ in size and conformation of loops that protrude from the central beta helix core. Therefore, a truncated version of Amb a 1, Cry j 1, and Cup a 1 was designed: lacking N- and C-terminal parts of the wild type protein, leaving the entire beta helix with the known T-cell epitopes.

FIGS. 6 and 7 show the differences in, sequence and structure between Amb a 1.3 alpha chain and Amb a 1.3 truncate. Protein expression of the different constructs was performed using E. coli BL21 (DE3) star in LBsupp media and incubation at 16° C. for 20 hours.

As depicted in FIG. 8, the Amb a 1.3 truncate construct with the Erwinia crysanthemi pelC leader sequence shows highest level of protein expression. Therefore, all following constructs (Cry j 1, Cup a 1, either full length or truncate versions) were cloned using the pelC leader.

### Patients IgE Binding

### DotBlot (see FIG. 9):

Patients Sera CE 2009, Patients 73, 74, 3, 60, 99, 38
Proteins: Amb Trunc Pel C fresh expressed, soluble lysate, Trunc content verified by SDS-Page, 2.9 mg/ml total protein, Trunc estimated 10% of total protein
Amb FL Pel C freshly expressed, soluble lysate, FL content verified by SDS-Page, 2.8 mg/ml total protein, FL estimated 10% of total protein
Amb alpha chain fresh expressed, 6M Urea, alpha chain content verified by SDS-Page, 5.7 mg/ml total protein, alpha chain estimated 10% of total protein
Amb R2 fresh expressed, 6M Urea, R2 content verified by

SDS-Page, 3.2 mg/ml total protein, R2 estimated 10% of total protein
BL21 induced with IPTG, no plasmid, soluble fraction
3.6 mg/ml total protein nat Amb a 1—conc. 0.2 mg/ml determined by Bradford assay

Spotted 50 μl per spot, sera were diluted 1:10, oN incubation at 4°, detection with anti human IgE AP BD Pharmingen 1:5.000, 4 h RT, substrate NBT/BCIP

Rabbit anti nat Amb a 1 dilution 1:10.000, oN incubation at 4°, detection with goat anti rabbit AP Jackson 1:10.000, 4 h RT, substrate NBT/BCIP

### ELISA (see FIG. 10)

Patients sera used: CE sera 5, 38, 60, 62, 74, 37, 48, 99

Coating conditions: 25 μg/ml total protein from bacterial lysates 2 μg/ml purified natural Amb a 1 (nAmb a 1)

RBL—Mediator Release Assay (See FIG. 11a-b)

### Proteins:

nat Amb a 1 or Cup a 1-0.2 mg/ml
Amb Trunc or Cup a 1 trunc:soluble lysate, 2.5 mg/ml total protein, Trunc estimated 10% of total protein
   BL21 induced with IPTG, no plasmid, soluble fraction, 3.5 mg/ml total protein

The protein concentration of nat Amb a 1 and nat Cup a 1 was 10 times less than the truncated variants, since in the extracts only approx. 10% Amb Trunc and Cup a 1 Trunc were present, respectively.

Only purified natural Amb a 1 and natural Cup a 1 could show mediator release. Truncated versions did not show mediator release, irrespective of whether they were expressed in E. coli or P. pastoris, thus reflecting the significant reduction in allergenicity. As control, BL21, no plasmid, soluble fraction was used.

Expression and Secretion of Polysaccharide Lyase Variant (Amb a 1.3 truncated) with Pichia pastoris

The P. pastoris clone with the specifically highest expression level (clone of Lane 8 in FIG. 14) was chosen for fermentation in a bioreactor (fermenter). After performing a pre-culture in shake flask (semi-synthetic medium), the culture was transferred into the fermenter containing 400 ml synthetic medium supplemented with trace elements. During the initial batch cultivation phase, P. pastoris was grown on glycerol as the carbon source in order to expand cell mass (biomass). Afterwards, the culture was subjected to nutrient addition (feeding) with concentrated glycerol solution in order to further increase cell biomass. After 4 hours of glycerol feeding, the expression of Amb a 1.3 truncated was induced by the addition of concentrated methanol (induction of AOX1 promoter). Standard cultivation conditions known in the art were applied (temperature=28° C. initially/24° C. during methanol induction; pH=5.8; dissolved oxygen saturation: 30%). At the end of cultivation (after ca. 100 h bioreactor cultivation time), the Amb a 1.3 truncated containing supernatant was seperated from cells by centrifugation (final wet cell mass ˜50% w/v). SDS-PAGE of fermentation supernatant is shown in FIG. 15. As indicated by the arrow in FIG. 15, Amb a 1.3 truncated was secreted at high titer.

### Example 2

### Recombinant Expression of Wild-Type Amb a 1 Leads to Significant Loss of Material Due to High Molecular Weight Aggregates

Recombinant wild-type Amb a 1 was expressed with E. coli BL21(DE3) cells harbouring a T7 promoter based expression vector. Cells were cultured in a bioreactor in LB-medium with magnesium salt and glucose. Recombinant protein expression induction was performed by the addition of IPTG. After 8 hours of cultivation, cells were harvested by centrifugation and afterwards lysed by freeze-thawing cycles. No Amb a 1 was detected in the soluble cell lysate (centrifugation supernatant). Extraction of recombinant Amb a 1 from the insoluble fraction (centrifugation pellet/inclusion body) was only possible by the application of strongly denaturing conditions (8 M urea+2-mercaptoethanol). Amb a 1 denatured extract was subjected to immobilized metal chelate affinity chromatography (IMAC). As shown in the upper part of the SDS-PAGE gel in FIG. 16, the fractions of the IMAC eluate contained considerable amounts of polymerized, high-molecular aggregates in the slots of the stacking gel of the SDS-PAGE. Similar results of strongly aggregated wild-type Amb a 1 were obtained with other chromatographic principles like hydrophobic interaction, ion exchange or size exclusion. The presence of high concentrations of urea was required during the whole purification process.

The high tendency to form insoluble aggregates lead to significant loss of yield and a lack of reproducibility of process output parameters, which made the process unsuitable for pharmaceutical large scale production.

1. A variant of a mature polysaccharide lyase having a beta helix structure, wherein said variant comprises an N- and/or C-terminal truncation and consists of at least 90% of the beta helix structure of the mature polysaccharide lyase.

2. The variant according to claim 1, wherein the N- and/or C-terminal truncation comprises at least 10 amino acid residues.

3. The variant according to claim 1, wherein the variant is hypoallergenic.

4. The variant of claim 1, wherein said N-terminal truncation comprises the region extending from the N-terminal end of the polysaccharide lyase to approximately 1 to 50 amino acid residues before the N-terminus of a first beta sheet being part of a beta helix structure and said C-terminal truncation comprises the region extending from the C-terminal end of the polysaccharide lyase to approximately 1 to 50 amino acid residues after the C-terminus of a last beta sheet part of the beta helix structure, such that the first and last beta sheet of the beta helix structure remains intact.

5. The variant of claim 1, wherein the polysaccharide lyase is a pectate lyase.

6. The variant of claim 5, wherein the allergen is selected from the group consisting of Amb a 1, Art v 6, Cha o 1, Cup a 1, Cry j 1 and Jun a 1.

7. The variant of claim 6, wherein the variant derived from Amb a 1 consists essentially of amino acid sequence SEQ ID No: 1, the variant derived from Cry j 1 consists essentially of amino acid sequence SEQ ID No: 2, the variant derived from Cup a 1 consists essentially of amino acid sequence SEQ ID No: 3, the variant derived from Jun a 1 consists essentially of amino acid sequence SEQ ID No: 4, the variant derived from Cha o 1 consists essentially of amino acid sequence SEQ ID No: 5 and the variant derived from Art v 6 consists essentially of amino acid sequence SEQ ID No: 6.

8. A polypeptide, comprising a secretion signal sequence fused directly or via a linker to the variant according to claim 1.

9. The polypeptide according to claim 8, wherein the secretion signal sequence is selected from the group consisting of a PelB, a PelC, a OmpA, a StII, an EX, a PhoA, a OmpF, a PhoE, a MalE, an OmpC, a LPP, a LamB, an OmpT, a LTB, a phosphatase (phol) and an invertase (Suc) secretion signal sequence and an α-mating factor prepro-sequence.

10. The polypeptide according to claim 8, wherein the secretion signal sequence is fused via a linker comprising a protease cleavage site.

11. The polypeptide according to claim 10, wherein the protease cleavage site is capable of being cleaved by thrombin, chymotrypsin, trypsin, pepsin, subtilisin, enterokinase, factor Xa, TEV protease and PreScission protease.

12. A nucleic acid molecule encoding a variant of a polysaccharide lyase according to claim 1.

13. The nucleic acid molecule according to claim 12, wherein the nucleotide sequence of said nucleic acid molecule is codon optimised and/or codon harmonised.

14. The nucleic acid molecule according to claim 12, wherein the nucleic acid molecule encoding the variant of Amb a 1 has the nucleotide sequence SEQ ID No: 7 or SEQ ID No: 8, the nucleic acid molecule encoding the variant of Cry j 1 has the nucleotide sequence SEQ ID No: 9 or 10, the nucleic acid molecule encoding the variant of Cup a 1 has the nucleotide sequence SEQ ID No: 11 or 12, the nucleic acid molecule encoding the variant of Art v 6 has the nucleotide sequence SEQ ID No: 13 or 14, the nucleic acid molecule encoding the variant of Jun a 1 has the nucleotide sequence SEQ ID No: 15 or 16 and the nucleic acid molecule encoding Cha o 1 has the nucleotide sequence SEQ ID No: 17 or 18.

15. A vector, comprising the nucleic acid molecule according to claim 12.

16. A method for the recombinant production of a polysaccharide lyase variant of claim 1, the method comprising:
a) cultivating a host cell comprising a nucleic acid molecule encoding the variant; and
b) isolating the polysaccharide lyase variant or polypeptide from a supernatant of the culture medium.

17. The method according to claim 16, wherein a secretion signal sequence is removed from the polysaccharide lyase variant by incubating the lyase variant of step b) to a protease capable of cleaving the secretion signal sequence from the lyase variant of a polypeptide.

18. The variant of claim 1, wherein the polysaccharide lyase is an allergenic pectate lyase.

19. A nucleic acid molecule encoding a variant of a polypeptide according to claim 8.

20. The nucleic acid molecule according to claim 19, wherein the nucleotide sequence of said nucleic acid molecule is codon optimised and/or codon harmonised.
